(19) United States
(12) Patent Application Publication
(10) Pub. No.: US 2013/0202526 A1
(43) Pub. Date: Aug. 8, 2013

(54) PAA NANOPARTICLES FOR PET IMAGING AND PDT TREATMENT

(71) Applicants: Ravindra K. Pandey, East Amherst, NY (US); Raoul Kopelman, Ann Arbor, MI (US); Anurag Gupta, Hamburg, NY (US); Munawwar Sajjad, Clarence Center, NY (US)

(72) Inventors: Ravindra K. Pandey, East Amherst, NY (US); Raoul Kopelman, Ann Arbor, MI (US); Anurag Gupta, Hamburg, NY (US); Munawwar Sajjad, Clarence Center, NY (US)

(73) Assignees: HEALTH RESEARCH, INC., Buffalo, NY (US); REGENTS OF THE UNIVERSITY OF MICHIGAN, Ann Arbor, MI (US); THE RESEARCH FOUNDATION OF STATE UNIVERSITY OF NEW YORK, Amherst, NY (US)

(21) Appl. No.: 13/566,426

(22) Filed: Aug. 3, 2012

Related U.S. Application Data
(63) Continuation-in-part of application No. 12/909,573, filed on Oct. 21, 2010.
(60) Provisional application No. 61/279,522, filed on Oct. 21, 2009.

(51) Int. Cl. A61K 41/00

(52) U.S. Cl. CPC A61K 41/0071
USPC 424/1.37; 424/1.29

(57) ABSTRACT

PAA nanoparticles containing at least one tetrapyrrolic photosensitizer and at least one PET imaging agent.

### CROSS-REFERENCE TO RELATED APPLICATIONS

This application is a continuation-in-part of U.S. patent application Ser. No. 12/909,573 filed Oct. 21, 2010 which in turn claims the benefit under 35 U.S.C. §119(e) of U.S. Provisional Application No. 61/279,522, filed Oct. 21, 2009, which applications are incorporated by reference herein in their entirety.

### STATEMENT REGARDING FEDERALLY SPONSORED RESEARCH OR DEVELOPMENT

This invention was made with government support under Grant Numbers CA19358 and CA114053 awarded by the National Institutes of Health. The government has certain rights in the invention.

### BACKGROUND OF THE INVENTION

The field of biomedical imaging has progressed tremendously from the discovery of X-ray to the imaging tools of today, such as magnetic resonance imaging, computed tomography, positron emission tomography and ultrasonography. The benefits of using these sophisticated noninvasive imaging tools are already evident. More accurate and timely diagnosis of disease has translated into improved patient care. Major new areas of research focus on development of the molecular, functional, cellular and genetic imaging tools of the future, aided by new information technology and image fusion/integration capabilities. Image guided therapy is growing rapidly. The development of multifunctional agents, which have the capability of using two or more imaging techniques that are complimentary to each other and can also be combined with a treatment modality, has great potential for significantly improving the outcome of patient treatment. The three critical components of image-guided therapy are navigation, control and monitoring the therapy delivery. They all rely on the target identification—Precise navigation requires clear identification of the target, to monitor treatment delivery, the target lesions and all adjacent tissues must be identified accurately while controlling the intervention of procedure.

Among the various cancer treatment modalities, photodynamic therapy (PDT) has shown encouraging clinical results for certain cancers. It involves the localization of certain therapeutic agents called photosensitizers (PS) into tumors upon systemic administration, which on exposing with an appropriate wavelength of light generates cytotoxic species (mainly singlet oxygen) believed to be responsible for cell death. Among the photosensitizers evaluated so far, the porphyrin-based compounds have shown a great potential in clinic or at advanced preclinical studies.

Nanoscience is being developed in conjunction with advanced medical science for further precision in diagnosis and treatment. Multidisciplinary biomedical scientific teams including biologists, physicians, mathematicians, engineers and clinicians are working to gather information about the physical properties of intracellular structures upon which biology's molecular machines are built. A new emphasis is being given to moving medical science from laboratory to the bedside and the community. This platform development program brings together an outstanding laboratory that is pioneering biomedical applications of PAA nanovectors (Kopelman), together with an innovative porphyrin chemistry and a world-class PDT group at RPCI that is highly experienced in the high volume screening and in vitro/in vivo evaluation of novel compounds, and in developing new therapies from the test tube to FDA approval for clinical use. Although nanoplatforms and nanovectors (i.e. a nanoplatform that delivers a therapeutic or imaging agent) for biomedical applications are still evolving, they show enormous promise for cancer diagnosis and therapy. The approach has been the subject of several recent reviews 2 Therapeutic examples include NP containing PDT agents, folate receptor-targeted, boron containing dendrimers for neutron capture and NP-directed thermal therapy. Recently, we have evaluated the therapeutic and imaging potential of encapsulated, post-loaded and covalently linked photosensitizer-NPs. In PAA NP the post-loading efficiency showed enhanced in vitro/in vivo therapeutic and imaging potential. PAA NP have core matrixes that can readily incorporate molecular or small NP payloads, and can be prepared in 10-150 nm sizes, with good control of size distributions. The surfaces of NPs can be readily functionalized, to permit attachment of targeting ligands, and both are stable to singlet oxygen (1O2) produced during PDT. PAA-NP have the advantages of (1) A relatively large knowledge base on cancer imaging, PDT, chemical sensing, stability and biodegradation. (2) No known in-vivo toxicity. (3) Long plasma circulation time without surface modification (see Preliminary Data), but with biodegradation and bioelimination rates controllable via the type and amount of selective cross-linking (introduced during polymerization inside reverse micelles). (4) Scale-up to 400 g material has been demonstrated, as well as storage stability over extended periods. Limitations include relative difficulty in incorporating hydrophobic compounds (although we have accomplished this), leaching of small hydrophilic components unless they are “anchored”, and unknown limitation on bulk tumor permeability because of hydrogel swelling.

The major challenge of cancer therapy is preferential destruction of malignant cells with sparing of the normal tissue. Critical for successful eradication of malignant disease are early detection and selective ablation of the malignancy. PDT is a clinically effective and still evolving locally selective therapy for cancers. The utility of PDT has been demonstrated with various photosensitizers for multiple types of disease. It is FDA approved for early and late stage lung cancer, obstructive esophageal cancer, high-grade dysplasia associated with Barrett's esophagus, age-related macular degeneration and actinic keratoses. PDT employs tumor localizing PSs that produce reactive 1O₂ upon absorption of light which is responsible for the destruction of the tumor. Subsequent oxidation-reduction reactions also can produce superoxide anions, hydrogen peroxide and hydroxyl radicals which contribute to tumor ablation 4. Photosensitizers have been designed which localize relatively specifically to certain subcellular structures such as mitochondria, which are highly sensitive targets 5. On the tumor tissue level, direct photodynamic tumor cell kill, destruction of the tumor supporting vasculature and possibly activation of the innate and adaptive anti-tumor immune system interact to destroy the malignant tissue 6. The preferential killing of the targeted cells (e.g. tumor), rather than adjacent normal tissues, is essential for PDT, and the preferential target damage achieved in clinical applications is a major driving force behind the use of the modality. The success of PDT relies on development of tumor-avid molecules that are preferentially retained in malignant cells but cleared from normal tissues. Clinical PDT initially was developed at Roswell Park Cancer Institute (RPCI), and we have one of the world's largest basic and clinical research programs. The RPCI group developed Photofrin®, the first generation FDA approved hematoporphyrin-based compound. Subsequently, our group has investigated structure activity relationships for tumor selectivity and photosensitizing efficacy, and used the information to design new PSs with high selectivity and desirable pharmacokinetics. Although the mechanism of porphyrin retention by tumors in not well understood, the balance between lipophilicity and hydrophilicity is recognized as an important factor 7 In our efforts to develop effective photosensitizers with the required photophysical characteristics, we used chlorophyll-a and bacteriochlorophyll-a as the substrates. Extensive QSAR studies on a series of the alkyl ether derivatives of pyropheophorbide-a (660 nm) led to selection of the best candidate, HPPH (hexyl ether derivative) 8,9, now in promising Phase II clinical trials. Our PS development now extends to purpurinimide (700 nm) and bacteriopurpurinimde (780-800 nm) series with high 1O2 producing capability 10-13 Long wavelength absorption is important for treating large deep-seated tumors, because longer wavelength light increases penetration and minimizes the number of optical fibers needed for light delivery within the tumor

Advantages of longer wavelength photosensitizers (700-800 nm) for phototherapy over HPPH. The penetration of light through tissue increases as its wavelength increases between 630 and 800 nm. Once light has penetrated tissue more than 2-3 mm it becomes fully diffuse (i.e. non-directional). In diffusion theory, the probability that a photon will penetrate a given distance into tissue is governed by the probability per unit path. The intrinsic absorption of most tissues is dominated by hemoglobin and deoxyhemoglobin, with the strong peaks of the absorption bands at wavelengths shorter than 630 nm. The tails of these bands extend beyond 630 nm and grow weaker with increasing wavelength. Thus the probability of a photon being absorbed by endogenous chromophores decreases with increasing wavelength from 630-800 nm and the scattering also decreases with wavelength 14 resulting in the very large increase in light penetration at ˜600 to 800 nm.

PDT and nanoparticle platforms. Photosensitizers have several very desirable properties as therapeutic agents deliverable by NP: (1) Only a very small fraction of administered targeted drug makes it to tumor sites and the remainder can cause systemic toxicity. However, PDT provides dual selectivity in that the PS is inactive in the absence of light and is innocuous without photoactivation. Thus the PS contained by the NP can be locally activated at the site of disease. (2) PDT effects are due to production of 1O2, which can readily diffuse from the pores of the NP (see Preliminary Data). Thus, in contrast to chemotherapeutic agents, release of encapsulated drug from the NP, is not necessary. Instead, stable NP with long plasma residence times can be used, which increases the amount of drug delivered to the tumors. (3) PDT is effective regardless of the intracellular location of the PS. While mitochondria are a principal target of 1O2, PS incorporated in lysosomes are also active the photodynamic process causes rupture of the lysosomes with release of proteolytic enzymes and redistribution of the PS within the cytoplasm. NP platforms also provide significant advantages for PDT: (1) High levels of imaging agents can be combined with the PS in the NP permitting a “see and treat” approach, with fluorescence image guided placement of optical fibers to direct the photoactivating light to large or subsurface tumors, or to early non clinically evident disease. (2) It is possible to add targeting moieties, such as cRGD or F3 peptide to the NP so as to increase the selective delivery of the PS. (3) The NP can carry large numbers of PS, and their surface can be modified to provide the desired hydrophilicity for optimal plasma pharmacokinetics. Thus, they can deliver high levels of PS to tumors, reducing the amount of light necessary for tumor cure.

F3 peptide is a 31-amino acid synthetic peptide derived from a fragment of the nuclear protein, high mobility group protein 2 (HMGN2)15. HMGN2 is a highly conserved nucleosomal protein thought to be involved in unfolding higher-order chromatin structure and facilitating the transcriptional activation of mammalian genes 62 when injected i.v., F3 peptide internalizes and accumulates in the nuclei of HL-60 cells and human MDA-MB-35 breast cancer cells. Tissue and cellular localization of F3 peptide indicated that it homes selectively to tumor blood vessels and tumor cells and has the remarkable property of being able to carry a payload into the cytoplasm and nucleus of the target cells. Furthermore, NPs with surface attached F3 behave similarly, attaching selectively to nucleolin expressing cells, and then channeled towards the cell nucleus. Recent literature shows that the F3 peptide binds to cell surface-expressed nucleolin on the target cells. Although primarily known as a nuclear and cytoplasmic protein a cell surface form of nucleolin also exists. Nucleolin is expressed on the surface of MDA-MB-35 cells and shuttles between the cytoplasm and the nucleus and between the cell surface and the nucleus. Nucleolin is also overexpressed in 9L glioma cells. Therefore, the mechanism of F3 targeting is recognition by nucleolin at the surface of actively growing cells (tumor cells and neovascular endothelial cells), which then binds and internalizes it, and transports it into the nucleus. While nucleolin can carry F3-targeted molecules from the cell surface into the nucleus, F3-labelled PAA nanoparticles containing Photofrin accumulated in the cytoplasm, which is useful because mitochondria are the primary target of PDT-produced 1O2. F3 targeting has been used recently to deliver nano-sized particles composed of lipids or quantum dots to tumor vasculature.

Integrins are a major group of cell membrane receptors with both adhesive and signaling functions. They influence behavior of neoplastic cells by their interaction with the surrounding extracellular matrix, participating in tumor development 16. Integrin αvβ3 in tumor cells binds to matrix metalloprotease-2 in a proteolytically active form and facilitates cell-mediated collagen degradation and invasion. It over-expresses in U87 and 9L glioma tumors. An increase in its expression is correlated with increased malignancy in melanomas. αvβ3 plays a critical role in angiogenesis and is up-regulated in vascular cells within human tumors. Significant overexpression of αvβ3 is reported in colon, lung, pancreas, brain and breast carcinomas, which was significantly higher in metastatic tumors. Our objective is to prepare a known integrin αvβ3-targeting ligand. While some recent work suggests that dimeric RGD peptides provide additional affinity and tumor binding, our recent in vitro data with HPPH-RGD conjugates (in one of which the binding site was blocked) shows the validity of our approach using monomeric RGD peptides.

Multiple, complementary techniques for tumor detection, including magnetic resonance, scintigraphic and optical imaging are under active development. Each approach has particular strengths and advantages. Optical imaging includes measurement of absorption of endogenous molecules (e.g. hemoglobin) or administered dyes, detection of bioluminescence in preclinical models, and detection of fluorescence from endogenous fluorophores or from targeted exogenous molecules. Fluorescence, the mission of absorbed light at a longer wavelength, can be highly sensitive: a typical cyanine dye with a lifetime of 0.6 nsec can emit up to 1032 photons/second/mole. A sensitive optical detector can image <103 photons/second. Thus even with low excitation power, low levels of fluorescent molecular beacons can be detected. A challenge is to deliver the dyes selectively and in high enough concentration to detect small tumors. Use of ICG alone to image hypervascular or “leaky” angiogenic vessels around tumors has been disappointing, due to its limited intrinsic tumor selectivity. Multiple approaches have been employed to improve optical probelocalization, including administering it in a quenched form that is activated within tumors, or coupling it to antibodies or small molecules such as receptor ligands. Recent studies have focused on developing dye conjugates of small bioactive molecules, to improve rapid diffusion to target tissue and use combinatorial and high throughput strategies to identify, optimize, and enhance in vivo stability of the new probes. Some peptide analogs of ICG derivatives have moderate tumor specificity and are entering pre-clinical studies. However, none of these compounds are designed for both tumor detection and therapy. It is important to develop targeting strategies that cope with the heterogeneity of tumors in vivo, where there are inconsistent and varying expressions of targetable sites.

Photosensitizers (PS) generally fluoresce and their fluorescence properties in vivo has been exploited for the detection of early-stage cancers in the lung, bladder and other sites 17 For treatment of early disease or for deep seated tumors the fluorescence can be used to guide the activating light. However, PS are not optimal fluorophores for tumor detection for several reasons: (i) They have low fluorescence quantum yields (especially the long wavelength photosensitizers related to bacteriochlorins). Efficient PS tend to have lower fluorescence efficiency (quantum yield) than compounds designed to be fluorophores, such as cyanine dyes because the excited singlet state energy emitted as fluorescence is instead transferred to the triplet state and then to molecular oxygen. (ii) They have small Stokes shifts. Porphyrin-based PS have a relatively small difference between the long wavelength absorption band and the fluorescence wavelength (Stokes shift), which makes it technically difficult to separate the fluorescence from the excitation wavelength. (iii) Most PS have relatively short fluorescent wavelengths, <800 nm, which are not optimal for detection deep in tissues.

In a separate study we have developed certain bifunctional conjugates that use tumor-avid PS to target the NIR fluorophores to the tumor 18. The function of the fluorophore is to visualize the tumor location and treatment site. The presence of the PS allows subsequent tumor ablation. The optical imaging allows the clinician performing PDT to continuously acquire and display patient data in real-time. This “see and treat” approach may determine where to treat superficial carcinomas and how to reach deep-seated tumors in sites such as the breast, lung and brain with optical fibers delivering the photo-activating light. A similar approach was also used for developing potential PDT/MRI conjugates in which HPPH was conjugated with Gd(III)DTPA Due to a significant difference between imaging and therapeutic doses, the use of a single molecule that includes both modalities is problematic. However, with PAA NPs we were able to solve this problem.

Positron emission tomography (PET) is a technique that permits non-invasive use of radioisotope labeled molecular imaging probes to image and assay biochemical processes at the level of cellular function in living subjects 20. PET predominately has been used as a metabolic marker, without specific targeting to malignancies. Recently, there has been growing use of radiolabeled peptide ligands to target malignancies. Currently, PET is important in clinical care and is a critical component in biomedical research, supporting a wide range of applications, including studies of gene expression, perfusion, metabolism and substrate utilization, neurotransmitters, neural activation and plasticity, receptors and antibodies, stem cell trafficking, tumor hypoxia, apoptosis and angiogenesis 21. Available isotope labels include 11C (t1/2=20.4 min), 18F (t1/2=110 min), 4Cu (t1/2=12.8 h) and 124I (t1/2=4.2 days). For targeting, a long circulation time may be desirable, as it can increase delivery of the agent into tumors. HPPH and the iodobenzyl pheophorbide-a have plasma half lives ˜25 h. The long radiological half life of 124I is well matched to the pheophorbides; it permits sequential imaging with time for clearance from normal tissue. Labeling techniques with radioiodine are well defined with good yield and radiochemical purity 22. Despite the complex decay scheme of 124I which results in only 25% abundance of positron (compared with 100% positron emission of 18F), in vivo quantitative imaging with 124I labeled antibodies has been successfully carried out under realistic conditions using a PET/CT scanner A variety of biomolecules have been labeled with 124I. We have devised a coupling reaction which rapidly and efficiently links 124I to a tumor-avid PS23-25, and used the conjugate to target and image murine breast tumor and its metastasis to lung (See Experimental Section). Acquisition of clinical PET images can be slow, but combination PET-CT scanners allow real time guidance of therapeutic interventions. Also, new developments in tracking may permit real time interventions guided by PET data sets.

NPs can optimize tumor detection and treatment of brain tumors. A photosensitizer (PS) with increased selectivity and longer wavelength could be a more suitable candidate for brain and deeply seated tumors (especially breast, brain and lung). The evolution of light sources and delivery systems is also critical to the progression of photodynamic therapy (PDT) in the medical field. Two different techniques: interstitial and intracavitary light delivery have been used for treatment of brain tumors. Powers et al. using interstitial PDT on patients with recurrent brain tumors showed that the majority of patients had tumor recurrence within two months of treatment. However, it was later observed that treatment failures appeared to occur outside the region of the effective light treatment. Chang et al reported an effective radius of tumor cell kill in 22 glioma patients of 8 mm compared with the 1.5 cm depth of necrosis noted by Pierria with the intracavitary illumination method. It is believed that tumor resection is important so that the numbers of tumor cells remaining to treat are minimized. With stereotactic implantation of fibers for interstitial PDT there is no cavity to accommodate swelling and a considerable volume of necrotic tumor which causes cerebral edema. However, cerebral edema can be readily controlled with steroid therapy. Compared to chemotherapy and radiotherapy, patients with brain tumors treated with PDT have definitely shown long-term survival, whereas glioma patients treated with adjuvant chemotherapy or radiotherapy do not show additional benefits as reported by Kostron et al. and Kaye et al. On the basis of our preliminary data, the αvβ3 targeted NPs may improve tumor-selectivity and PDT outcome.

Importance of multifunctional NPs in brain-tumor imaging and PDT. The prognosis for patients with malignant brain tumors is linked to the completeness of tumor removal. However, the borders of tumors are often indistinguishable from surrounding brain tissue so tumor excision is highly dependent upon the neurosurgeon's judgment. To identify tumors, neurosurgeons use diagnostic imaging methods such as Computed Tomography (CT) or Magnetic Resonance Imaging (MRI), which enhance the contrast between tumor and surrounding brain tissue. However, there are frequently discrepancies between intraoperative observations of tumor margins and preoperative diagnostic imaging studies. Unlike CT and MRI, intraoperative ultrasound can provide real-time information to locate the tumor and define its volume. However, once resection commences is also limited by signal artifacts caused by blood and surgical trauma limit tumor identification at the resection margin. Intraoperative MRI allows the neurosurgeon to obtain images during surgery, which can improve the completeness of the tumor resection, however microscopic disease is still not detected. In an ideal situation, the surgeon would perform the brain tumor resection with continuous guidance from high-contrast fluorescence from the tumor observed directly in the resection cavity.

### BRIEF SUMMARY OF THE INVENTION

The present invention relates to polyacrylic acid (PAA) nanoparticles containing a photosensitizer and an imaging enhancing agent. The imaging agent is preferably a PET imaging agent and more preferably an ¹²⁴I labeled compound. The photosensitizer is preferably selected from chlorins, bacteriochlorins, pyropheophorbides, and mixtures thereof. The nanoparticles preferably contain at least one photosensitizer comprises a moiety containing ¹²⁴I and also acts as an imaging agent. The photosensitizer and imaging agent are preferably post loaded onto the nanoparticle after nanoparticle formation.

The photosensitizer is preferably a tetrapyrollic photosensitizer having the structural formula:

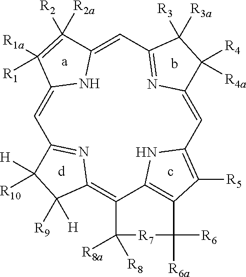

or a pharmaceutically acceptable derivative thereof, wherein:

R₁ and R₂ are each independently substituted or unsubstituted alkyl, substituted or unsubstituted alkenyl, —C(O)Rₐ or —COORₐ or —CH(CH₃)(ORₐ) or —CH(CH₃)(O(CH₂)ₙXRₐ) where Rₐ is hydrogen, substituted or unsubstituted alkyl, substituted or unsubstituted alkenyl, substituted or unsubstituted alkynyl, or substituted or unsubstituted cycloalkyl; where R₂ may be —CH═CH₂, —CH(OR₂₀)CH₃, —C(O)Me, —C(═NR₂₁)CH₃ or —CH(NHR₂₁)CH₃

where X is an aryl or heteroaryl group;

n is an integer of 0 to 6;

where R₂₀ is methyl, butyl, heptyl, docecyl or 3,5-bis(trifluoromethyl)-benzyl; and

R₂₁ is 3,5-bis(trifluoromethyl)benzyl;

R₁ₐ and R₂ₐ are each independently hydrogen or substituted or unsubstituted alkyl, or together form a covalent bond;

R₃ and R₄ are each independently hydrogen or substituted or unsubstituted alkyl;

R₃ₐ and R₄ₐ are each independently hydrogen or substituted or unsubstituted alkyl, or together form a covalent bond;

R₅ is hydrogen or substituted or unsubstituted alkyl;

R₆ and R₆ₐ are each independently hydrogen or substituted or unsubstituted alkyl, or together form ═O;

R₇ is a covalent bond, alkylene, azaalkyl, or azaaraalkyl or ═NR₂₀ where R₂₀ is 3,5-bis(tri-fluoromethyl)benzyl or —CH₂X—R¹ or —YR¹ where Y is an aryl or heteroaryl group;

R₈ and R₈ₐ are each independently hydrogen or substituted or unsubstituted alkyl or together form ═O;

R₉ and R₁₀ are each independently hydrogen, or substituted or unsubstituted alkyl and R₉ may be —CH₂CH₂COOR² where R² is an alkyl group that may optionally substituted with one or more fluorine atoms;

each of R₁-R₁₀, when substituted, is substituted with one or more substituents each independently selected from Q, where Q is alkyl, haloalkyl, halo, pseudohalo, or —COOR_{b} where R_{b} is hydrogen, alkyl, alkenyl, alkynyl, cycloalkyl, aryl, heteroaryl, araalkyl, or OR_{c} where R_{c} is hydrogen, alkyl, alkenyl, alkynyl, cycloalkyl, or aryl or CONR_{d}Rₑ where R_{d} and Rₑ are each independently hydrogen, alkyl, alkenyl, alkynyl, cycloalkyl, or aryl, or NR_{f}R_{g} where R_{f} and R_{g} are each independently hydrogen, alkyl, alkenyl, alkynyl, cycloalkyl, or aryl, or ═NRₕ where Rₕ is hydrogen, alkyl, alkenyl, alkynyl, cycloalkyl, or aryl, or is an amino acid residue;

each Q is independently unsubstituted or is substituted with one or more substituents each independently selected from Q₁, where Q₁ is alkyl, haloalkyl, halo, pseudohalo, or —COOR_{b} where R_{b} is hydrogen, alkyl, alkenyl, alkynyl, cycloalkyl, or aryl, heteroaryl, araalkyl, or OR_{c} where R_{c} is hydrogen, alkyl, alkenyl, alkynyl, cycloalkyl, or aryl or CONR_{d}Rₑ where R_{d} and Rₑ are each independently hydrogen, alkyl, alkenyl, alkynyl, cycloalkyl, or aryl, or NR_{f}R_{g} where R_{f} and R_{g} are each independently hydrogen, alkyl, alkenyl, alkynyl, cycloalkyl, or aryl, or ═NRₕ where Rₕ is hydrogen, alkyl, alkenyl, alkynyl, cycloalkyl, or aryl, or is an amino acid residue.

The photosensitizer is preferably a chlorophyll-based photosensitizer post-loaded to biodegradable and biocompatible polyacrylamide (PAA) nanoparticles.

The photosensitizer may be conjugated with an image enhancing agent prior to incorporation into the nanoparticle, after incorporation into the nanoparticle or the photosensitizer and/or image enhancing agent may chemically bound to the nano particle and/or one or more of the photosensitizer and image enhancing agent may be physically bound to the nanoparticle.

Imaging enhancing agents may be for essentially any imaging process, e.g. Examples of such imaging enhancing agents are discussed in the background of the invention previously discussed and in the list of references incorporated by reference herein as background art.

It is to be understood that other agents may be incorporated into the nanoparticle such as tumor targeting moieties and tumor inhibiting or tumor toxic moieties.

The utility of a biodegradable/biocompatible, nontoxic polyacrylamide-based nanoparticles-photosensitizer formulation for developing highly efficient “Multimodality Platform” for tumor-imaging by PET and photodynamic therapy is described. Comparative tumor-imaging, biodistribution and PDT efficacy data clearly demonstrate the advantages of our invention

### BRIEF DESCRIPTION OF THE SEVERAL VIEWS OF THE DRAWINGS

FIG. 1A shows the structural formula of HPPH-CD (cyanine dye) conjugate used as a photosensitizer and imaging agent.

FIG. 1B is a graph showing in vivo photosensitizing efficacy of HPPH-CD conjugate 1 in C3H mice bearing RIF tumors (10 mice/group) at variable drug doses. The tumors were exposed to light (135 J/cm2/75 mW/cm2) at 24 h post-injection.

FIG. 1C shows a scanned image showing localization of the conjugate 1 in a live mouse 24 h after injection (drug dose 0.3 μmole/kg). (Without PAA NP0).

FIG. 2 shows whole body images of BALB/c mice bearing Colon26 tumors with PAA NPs formulations (HPPH and cyanine dye (CD) were post-loaded in 2 to 1 ratio). The CD concentration was kept constant (0.3 μmol/kg) at the images were obtained at variable time points. A=24 h, B=48 h and C=72 h post injection (λex: 785 nm; λEm: 830 nm). L=Low and H=High.

FIG. 3 is a graph showing in vivo PDT efficacy of HPPH and CD post loaded in a ratio of 2:1 and 4:1 in PAA and ORMOSIL NPs. Note: HPPH dose: 0.47 μmol/kg in PAA NPs and 0.78 μmol/kg in ORMOSIL NPs.

FIG. 4. Slow release of HPPH and CD from PAA NPs (post loaded in 2:1 ratio) after several washes with 1% HSA.

FIG. 5A is a diagram showing structure of PAA nanoparticles (PAA NP's).

FIG. 5B shows comparative in vivo imaging at variable time points of BALB/c mice bearing Colon26 tumors with HPPH-CD conjugate 1 and CD-conjugated with PAA NPs/post; -loaded with HPPH. The NPs were more tumor specific. (Mouse 1)

FIG. 6 shows a series of scans wherein Panel 1 (4T1 tumors): Primary (PT) and metastasized tumors (MT) dissected and Panel 2 (4T1 tumors): PET imaging of the dissected primary and metastasized tumors. Panel 3 (BALB/C mouse bearing 4T1 tumor): Whole body PET imaging. The tumor metastasis in lung was clearly observed. Panel 4: The position of the lung is shown by the transmission scan using 57Co source in mice with no lung metastasis. Panel 5: (BALB/C mouse bearing Colo-26 (non-metastatic tumor): Whole body imaging by PET. A high accumulation of the 124I-photosensitizer in tumor is clearly observed without any significant accumulation in lungs (injected dose: 100 μCi). T=Tumor, PT=Primary tumor; MT=Metastatic tumor.

FIG. 7. In vivo biodistribution of 18F-FDG (100 μCi, half-life 2 h) at 110 min and 124I-PS 2 (100 μCi, half-life 4.2 d) at 48 h in BALB/c mice bearing Colon 26 tumor (3 mice/group). Tumor-uptake was similar for both agents. However, the higher uptake of FDG over 124I-PS 2 in normal organs is clearly evident.

FIG. 8A shows in vivo comparative in vivo PET imaging (72 h post injection) and biodistribution (24 h, 48 h and 72 h post injection) of 124I-labeled photosensitizer 2 without PAA nanoparticles in BALB/c mice bearing Colon26 tumors (see the text). (Biodistribution of PET imaging agent 2: No PAA, with PAA).

FIG. 8B shows in vivo comparative in vivo PET imaging (72 h post injection) and biodistribution (24 h, 48 h and 72 h post injection) of 124I-labeled photosensitizer 2 with PAA nanoparticles in BALB/c mice bearing Colon26 tumors (see the text). (Biodistribution of PET imaging agent 2: No PAA, with PAA).

FIG. 8C shows biodistribution of PET imaging agent 2, no PAA and with PAA.

FIG. 9. Fluorescence intensity of cells targeted by F3-targeted (A series), F3-Cys targeted (B series) and nontargeted NPs (F series) in nucleolin rich MDA-MB-435 cell lines.

FIG. 10. Fluorescence (left) & Live/dead cell assay (right) of HPPH conjugated PAA NPs + or −F3-Cys peptide incubated for 15 min with MDA-MB-435 cells.

FIG. 11. Confocal images showing the target-specificity of F3-Cys peptide in 9L Glioma tumor cells. Left: F3-Cys PEG Rhodamine-PAA NPs (9L cells). Right: PEG Rhodamine-PAA NPs (9L Cells).

FIG. 12. In vivo biodistribution of ¹⁴C-labeled HPPH, and ¹⁴C-labeled HPPH post-loaded into PAA NPs in BALB/c mice bearing Colon26 tumors. ¹⁴C-labeled PS (3.8 μCi/0.2 mL) were administered to 12 mice/group. At 24, 48, 72 h after injection, three mice/time-point were sacrificed. The organs of interest were removed and the radioactivity was measured. The raw data were converted to counts/gram of tissue.

FIG. 13A shows In vivo biodistribution of iodinated photosensitizer at 24, 48 and 72 h post injection.

FIG. 13B shows In vivo biodistribution of iodinated photosensitizer using variable sizes of PAA NPs at 24, 48 and 72 h post injection 531-ME Post-Loaded into 30 nm PAA Nanoparticles.

FIG. 13C shows In vivo biodistribution of iodinated photosensitizer using variable sizes of PAA NPs at 24, 48 and 72 h post injection 531-ME Post-Loaded into 150 nm PAA Nanoparticles.

FIG. 14 shows the structural formula of HPPH.

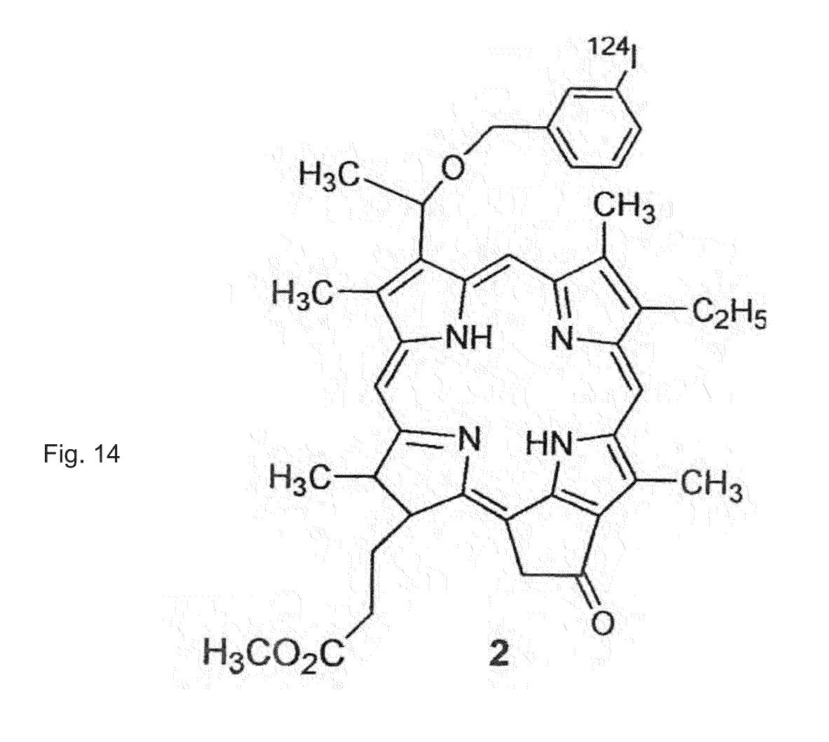

FIG. 15 is a diagram of Multifunctional PAA Nanoparticles.

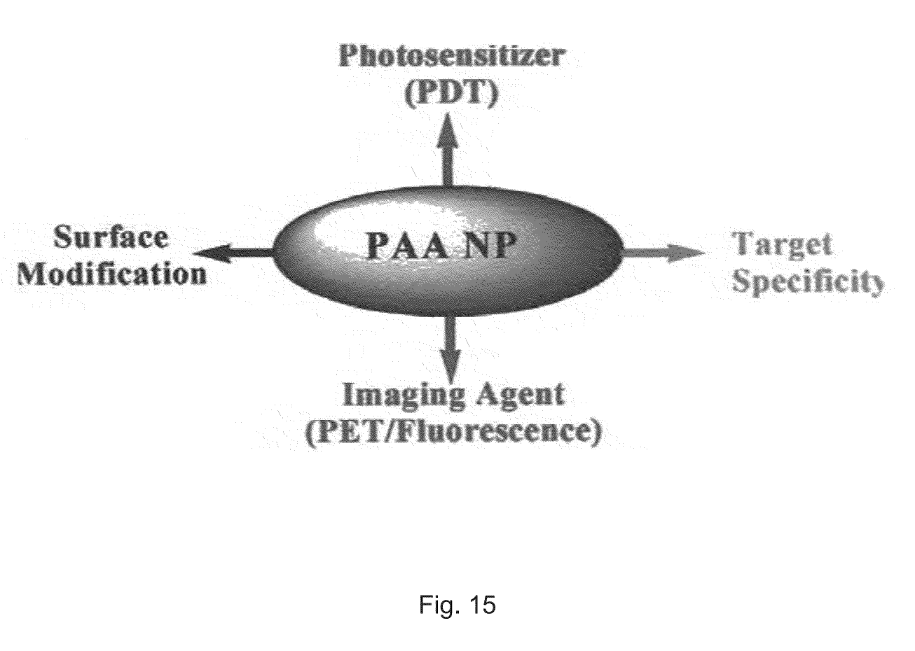

FIG. 16 shows flow diagrams for preparation of postloaded nanoparticles.

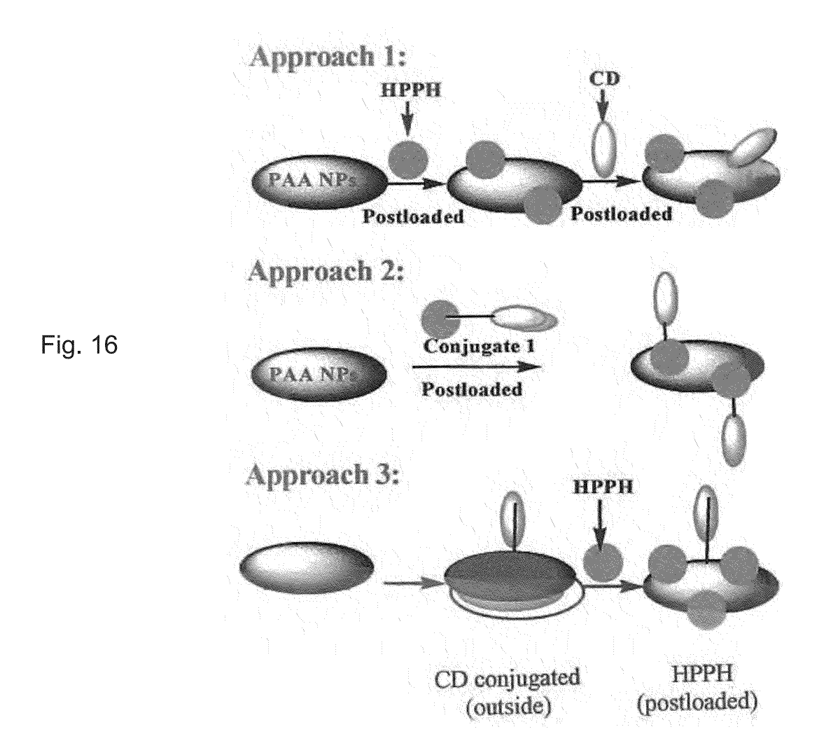

FIG. 17A shows the structure of photosensitizer 1(PS1).

FIG. 17B shows the corresponding ¹²⁴I-labeled analog 2 of PS1.

FIG. 17C shows the structure of ¹⁸F-fluoro-deoxyglucose (FDG).

FIG. 17D shows a schematic representation of photosensitizer (1 or 2) post-loaded in polyacrylamide (PAA) nanoparticles (NP1).

FIG. 18A is a curve showing electronic absorbance spectra for NP1 at various times during the post-loading procedure.

FIG. 18B shows fluorescence spectra of NP1 at various times during the post-loading procedure.

FIG. 18C shows electronic absorbance spectra for PS1 and NP1 in drug solution form (aqueous tween-80 solution) and in 17% Bovine Calf Serum in PBS (BCS-PBS).

FIG. 18D shows the fluorescence spectra for PS1, NP1, PS1 in 17% BCS-PBS and NP1 in 17% BCS-PBS. The concentration for photosensitizer 1 in all samples is three μM and the concentration of Tween-80 is less than 1%.

FIG. 19A shows whole body PET Images of BALB/c mice bearing subcutaneous Colon26 rumors on the light shoulder with ¹²⁴I-PS2 at 24, 48, and 72 h post-injection (i.v.).

FIG. 19B shows whole body PET Images of BALB/c mice bearing subcutaneous Colon26 rumors on the light shoulder with ¹²⁴I-PS2 post-loaded in PAA NPs (NP2) at 24, 48, and 72 h post-injection (i.v.).

FIG. 19C is a bar graph showing relative uptake values (RUV) of PS2 and NP2.

FIG. 20A is a bar graph showing comparative in-vivo biodistribution of (A): NP2, PS2 and FDG at 90 minutes post injection in BALB/c mice (3 mice/group) bearing subcutaneous Colon26 tumors on the right shoulder

FIG. 20B is a bar graph showing comparative in vivo biodistribution of NP2 at 24, 48 and 72 hours post injection in BALB/c mice (3 mice/group) bearing subcutaneous Colon 26 tumors on the right shoulder.

FIG. 20C is a table showing the ratio of tumor to various organs/tissues/fluids for PS2, NP2 at 24 h, and for ¹⁸F-FDG at 90 min post-injection in BALB/c mice (3 mice/group) bearing subcutaneous Colon26 tumors on the right shoulder.

FIG. 21 shows whole-Body fluorescence reflectance images of BALB/c mice bearing subcutaneous Colon26 tumors. (A-C): PS1, (D-F): NP1 at 24, 48, and 72 h post-injection (i.v.). The λ_{Exc}=665 nm and the λ_{Em}≧700 nm.

FIG. 22A shows in vivo PDT data (% mice cured) by Kaplan-Meier survival curve show a significant difference in PDT efficacy of PS1 with and without NPs at a dose of 1.0 μmole/kg. The tumors were exposed to light (135 J/cm² and 75 mW/cm²) 24 h post-injection. The P value for the for the two survival curves is <0.0001 as determined by the Mantel-Cox test.

FIG. 22B is a curve showing the weights of BALB/c mice (3 mice/group) injected with 100 mg/kg or 400 mg/kg of blank PAA NPs, recorded daily for 29 days.

FIG. 23 shows formalin-fixed, paraffin embedded hematoxylin-eosin (H.E.) stained tissue sections (representative sample for 400 mg/kg): (a) Liver, (b) spleen, (c) heart, (d) kidney, and (e) lung [Magnification: 200×].

FIG. 24A is a distribution curve characterizing of the size of the blank PAA nanoparticle formulation used for Photodynamic Therapy/fluorescence reflectance imaging and toxicology studies. The mean diameter is 30 nm.

FIG. 24B is a distribution curve characterizing of the size of NP1 in Tween-80/PBS (concentration of Tween-80 is <1%). The mean diameter is 35.1 nm.

FIG. 25 shows biodistribution of PS2: 24, 48, and 72 H post-injection.

FIG. 26 shows the release profile of PS1 from NP1.

### DETAILED DESCRIPTION OF THE INVENTION

Application of HPPH, a tumor-avid photosensitizer for developing bifunctional agents for fluorescence imaging/PDT and its limitations:

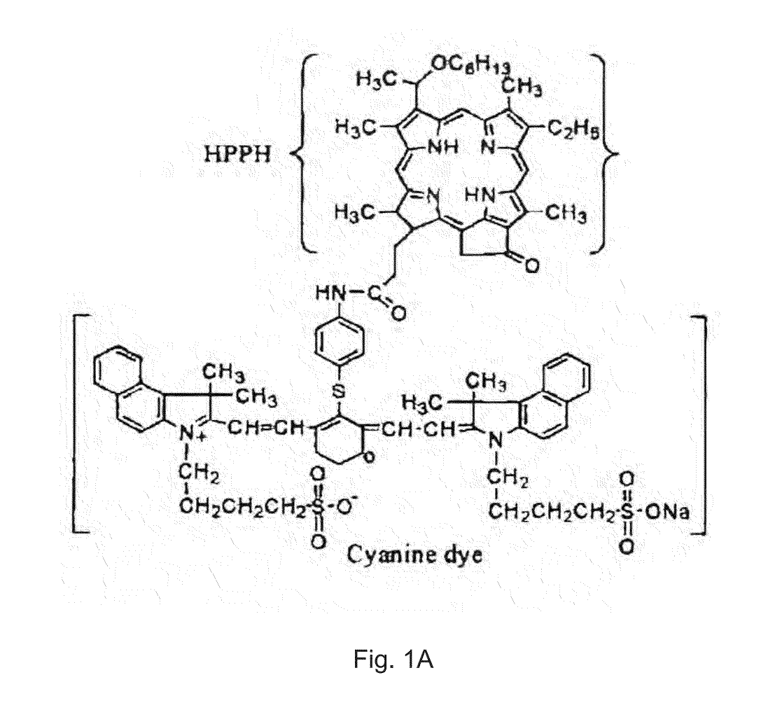

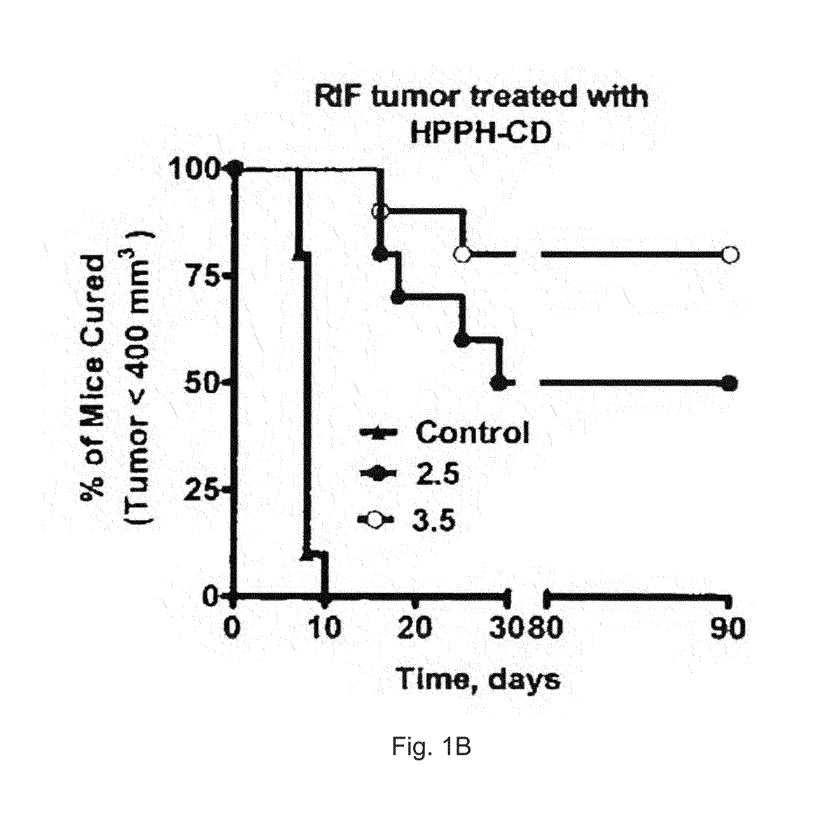

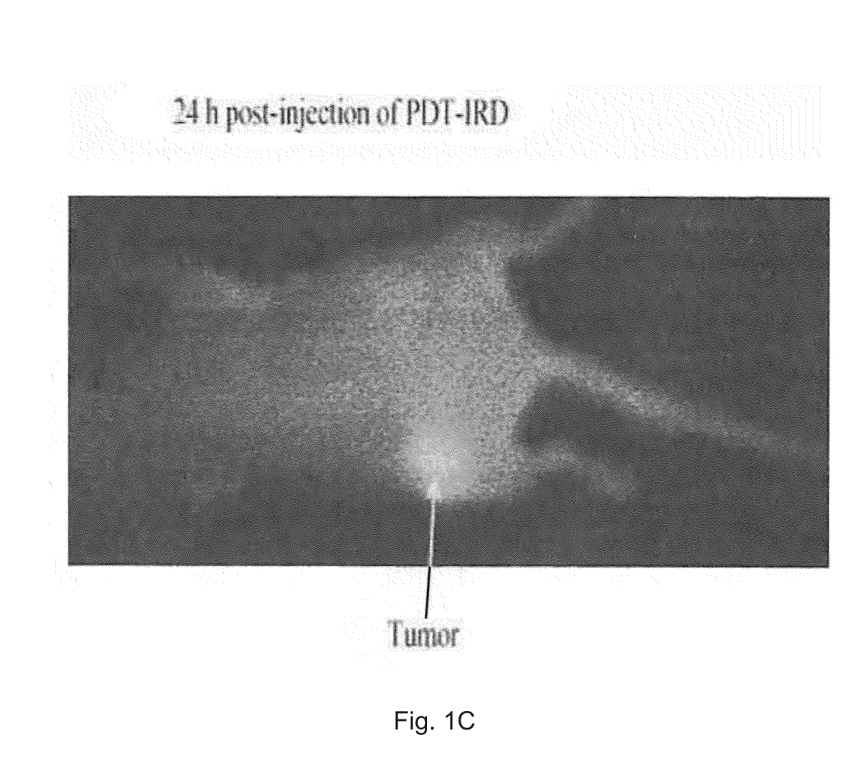

We have previously shown that certain tumor-avid PS(s) (e.g., HPPH) conjugated with NIR absorbing fluorophore(s) (non-tumor specific cyanine dyes) can be used as bifunctional agents for tumor-imaging by fluorescence and phototherapy (PDT). Here, HPPH was used as a vehicle to deliver the imaging agent to tumor. The limitation of this approach was that the conjugate exhibited significantly different dose requirements for the two modalities. The imaging dose was approximately 10-fold lower than the phototherapeutic dose (FIGS. 1B and 1C), which could be due to a part of the 1O2 (a key cytotoxic agent responsible for the destruction of the tumors) produced on exciting the PS being quenched by the fluorophore leading to its photo-destruction. Exposing the tumor at 780 nm (excitation wavelength for the cyanine dye) produced in vivo emission at 860 nm and, as expected, no significant photobleaching of the fluorophore (CD) or the PS(HPPH) was observed.

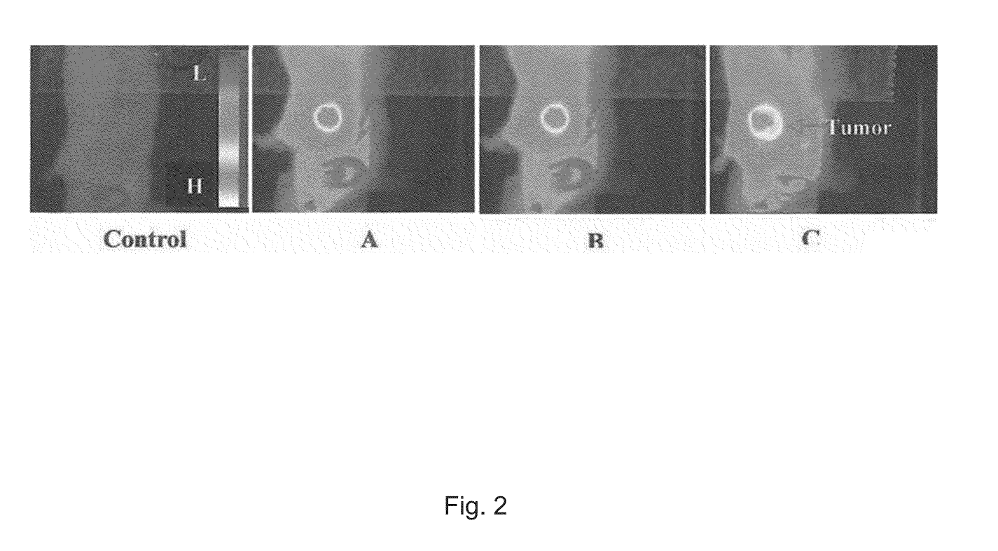

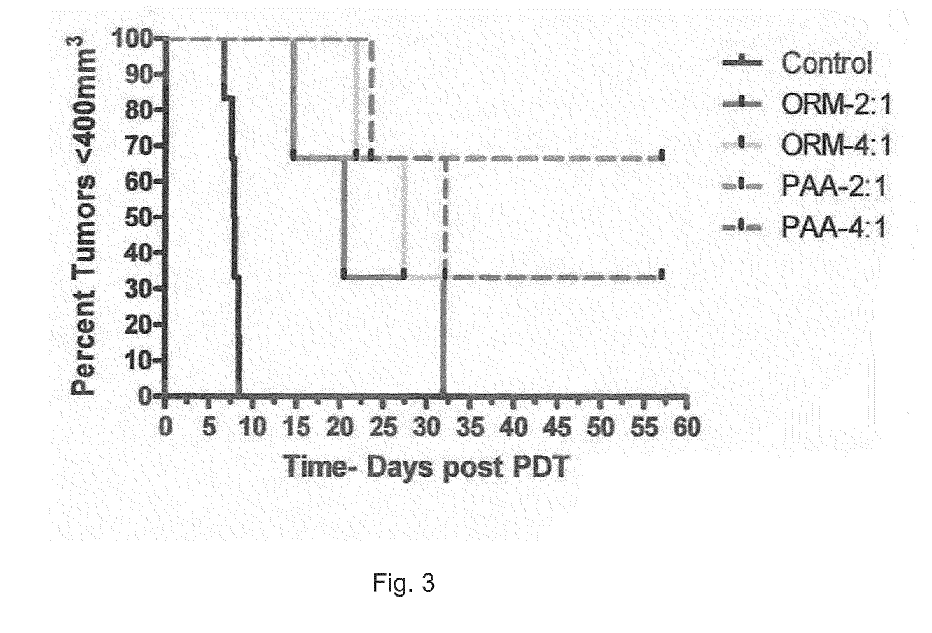

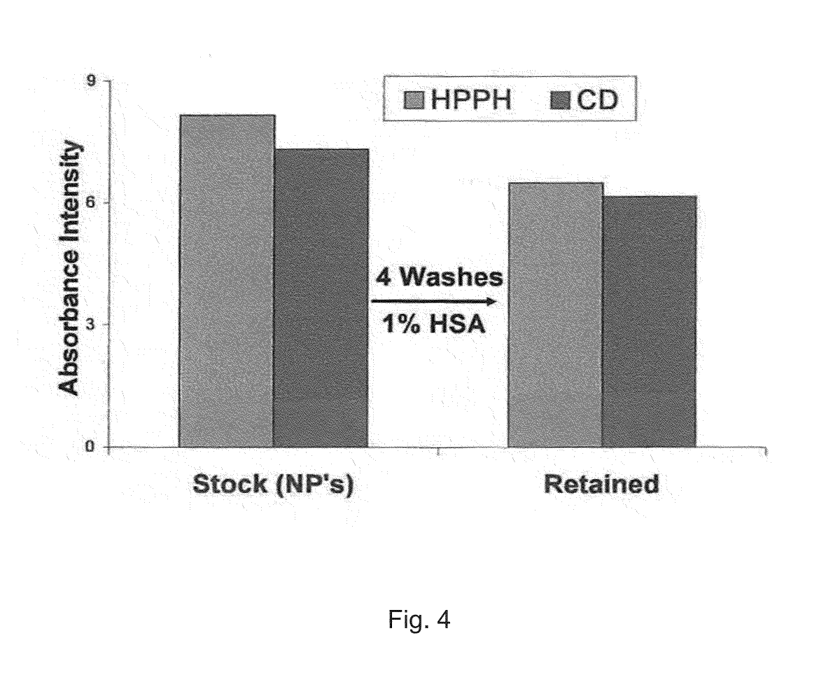

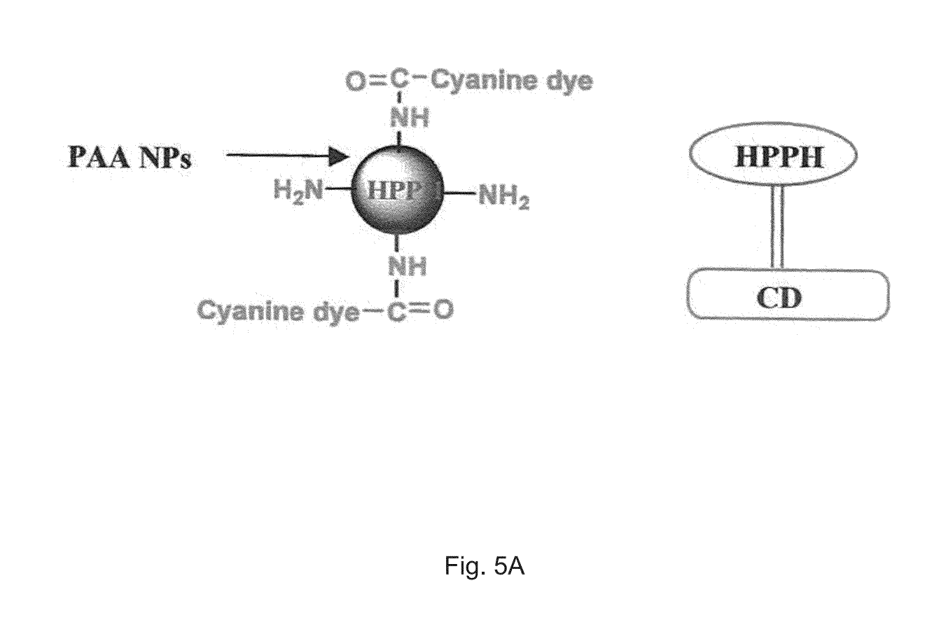

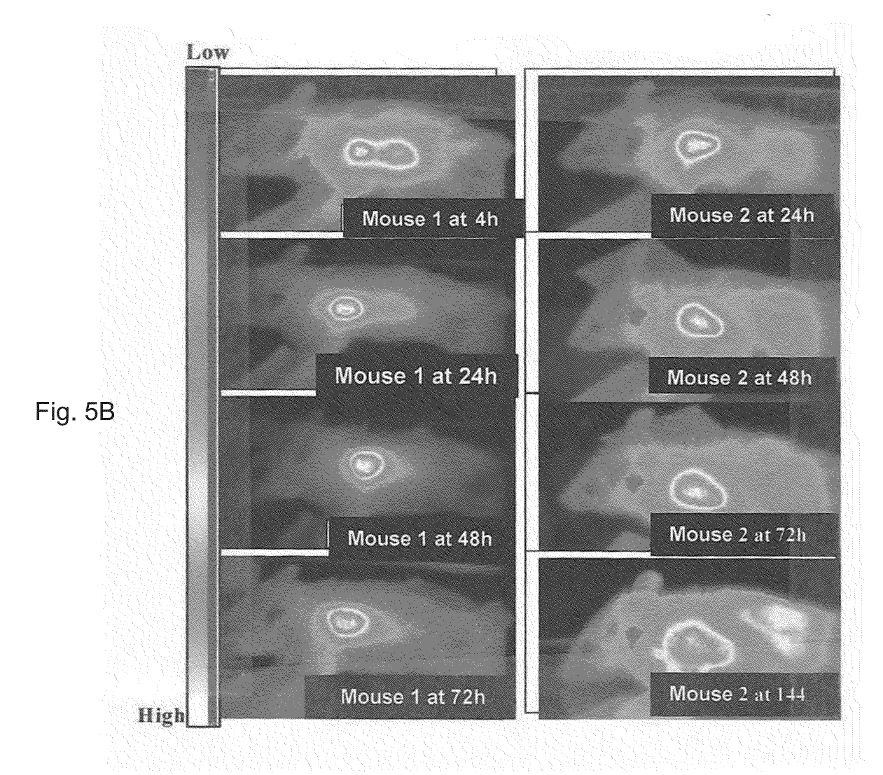

For investigating the utility of PAA NPs three different approaches were used. First HPPH and the cyanine dye (fluorophore) were post-loaded in variable ratios (HPPH to CD: 1:1; 2:1; 3:1 and 4:1 molar concentrations). In brief, HPPH was postloaded to PAA NPs first. Free HPPH was removed by spin filtration and then cyanine dye was postloaded. It was spin-filtered again, washed several times with 1% bovine calf serum and the concentration was measured. The 2:1 formulations produce the best tumor imaging and long-term tumor cure in BALB/c mice bearing Colon26 tumors. This formulation contained in a single dose the therapeutic dose of HPPH (0.47 mmol/kg) and the imaging dose of Cyanine dye (0.27 mol/kg), which were similar to the components used alone for tumor imaging and therapy, but with much more tumor selectivity (skin to tumor ratio of HPPH was 4:1 instead of 2:1 without NPs). Under similar treatment parameters the ORMOSIL NPs showed a significantly reduced response (imaging and PDT, not shown). The stability of the drugs in PAA NP was established by repeated washing with aqueous bovine calf serum through AMICON centrifugal filter units with a 100 KDa or larger cut off membrane and drug in the filtrate was measured spectrophotometrically. The comparative in vivo PDT efficacy of the ORMOSIL and PAA formulations, their tumor imaging potential and stability (in vitro release kinetics) is shown in FIGS. 2-4, which clearly illustrate the advantages of PAA NPs in reducing the therapeutic dose by almost 8-fold without diminishing the tumor-imaging potential and also avoiding the Tween-80 formulation required for the HPPH-CD conjugate 1. In the 2^{nd} approach the HPPH CD conjugate 1 was post-loaded to PAA NPs, which certainly enhanced the tumorimaging, but the therapeutic dose was still 10-fold higher (similar to the HPPH CD conjugate, FIG. 5B). In the 3rd approach the cyanine dye was conjugated peripherally to the PAA NPs first and then HPPH was post loaded. Again, compared to HPPH-CD conjugate 1, the PAA formulation showed enhanced tumor-specificity (imaging) (FIG. 5B).

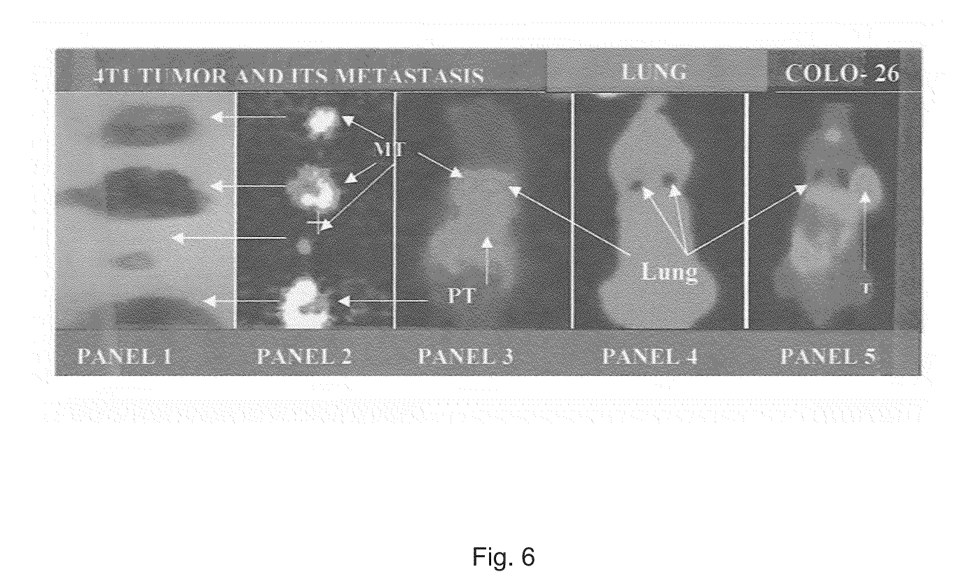

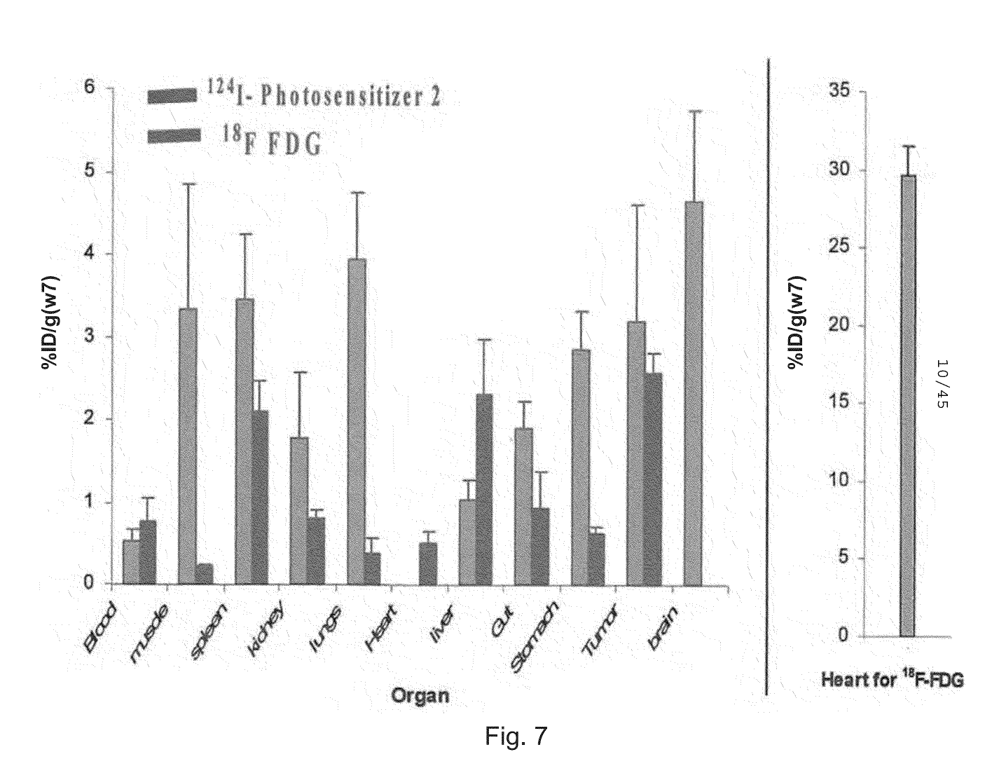

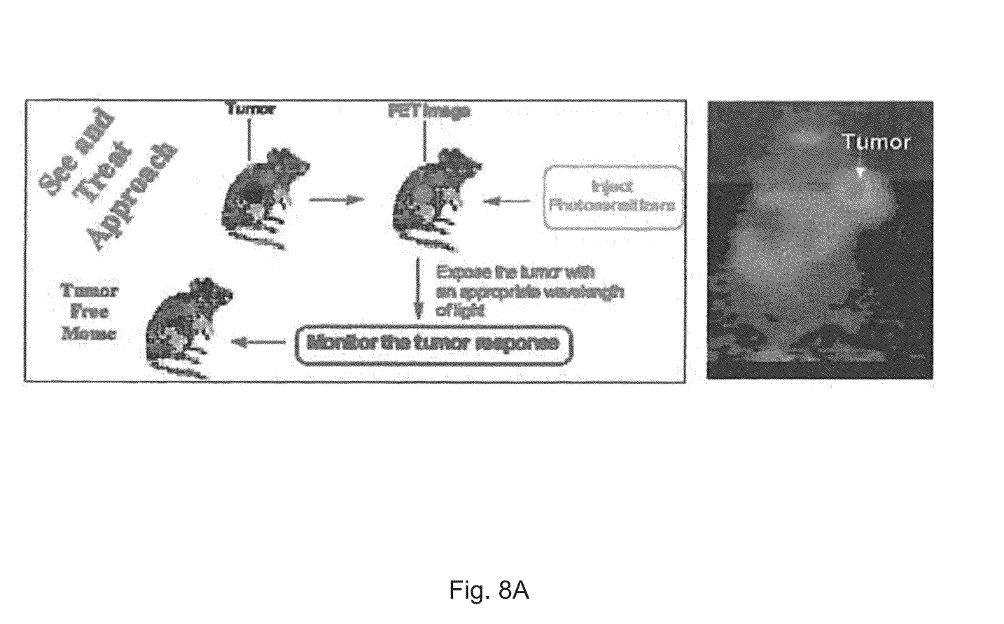

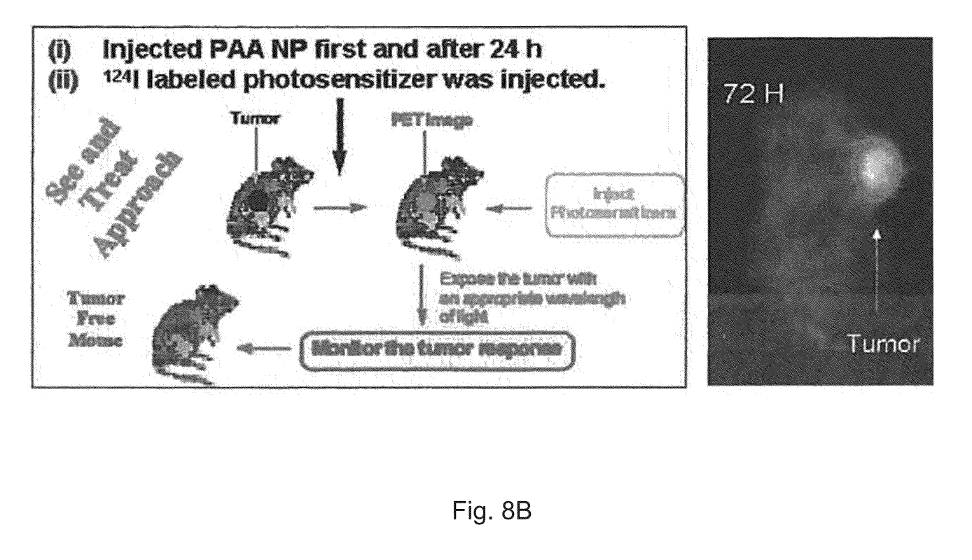

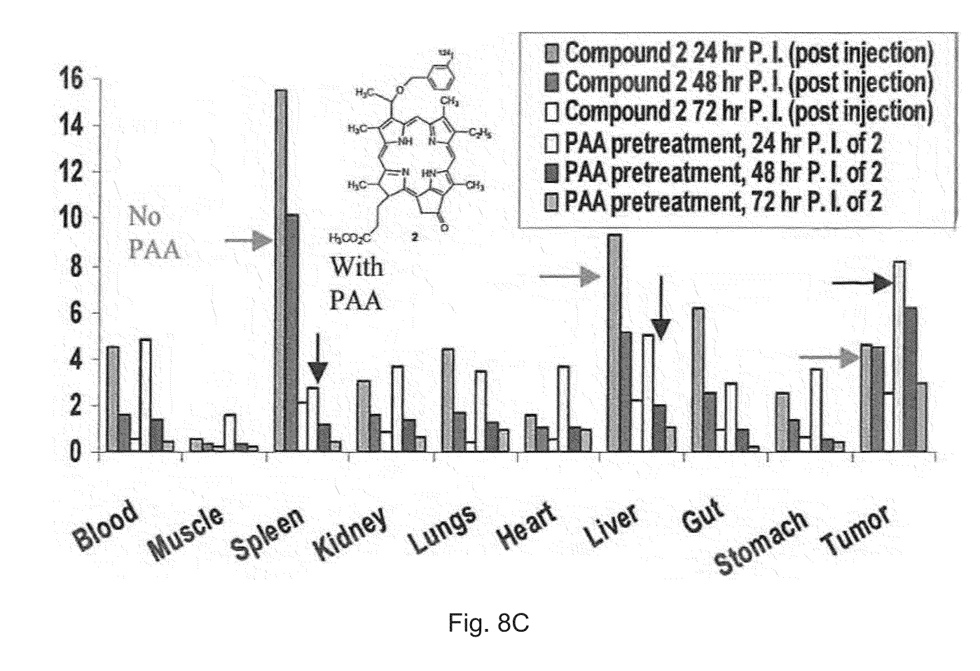

PET imaging and PDT: PAA NPs decreased the liver uptake of the 124I-photosensitizer (PET imaging agent) and enhanced the tumor-specificity. Our initial investigation with an 124I-labeled PS 2 indicates its in vivo PDT efficacy and capability of detecting tumors 104-106 (RIF, Colon26, U87, GL261, pancreatic tumor xenograft) and tumor metastases (BALB/c mice bearing orthotopic 4T1 (breast) tumors) (FIG. 6). Interestingly, compared to 18F FDG PS 2 showed enhanced contrast in most of the tumors including those where 18F FDG-PET provides limited imaging potential (e.g., brain, lung and pancreatic tumors). See FIG. 7 for comparative biodistribution. This is the first report showing the utility of porphyrin-based compounds as a “BIFUNCTIONAL AGENT” for imaging breast tumor and tumor metastasis. Similar to most NPs, PAA NP accumulate in liver and spleen. Their clearance rate from most organs is significantly faster than Ormosil NP and they do not show long-term organ toxicity. Even tumor-avid porphyrinbased PS exhibit high uptake in liver and spleen, but are non-toxic until exposed to light. The PS clear from the system quickly (days) without organ toxicity. However, radioactive PS such as the 124I-labeled analog 2 (superior to 18F-FDG in PET-imaging of lung, brain, breast and pancreas tumors) with a T1/2 of 4.2 days could cause radiation damage to normal organs. Based on the observation of high uptake of PAA NPs in liver and spleen (below) we postulated that saturating the organs with the non-toxic PAA NPs before injecting the PET agent might reduce uptake and radiation damage by 124I-imaging agent. For proof-of principle blank PAA NPs were first injected (i.v.) into mice bearing Colon26 tumors followed 24 h later by i.v. 124I-analog (100-50 μCi). The mice were imaged at 24, 48 and 72 h post injection and biodistribution studies were performed at each time point summarized in FIGS. 8A and 8C (only 72 h images shown).

The presence of PAA NPs made a remarkable difference in tumor contrast with brain, lung and pancreatic tumors). See FIG. 7 for comparative biodistribution.

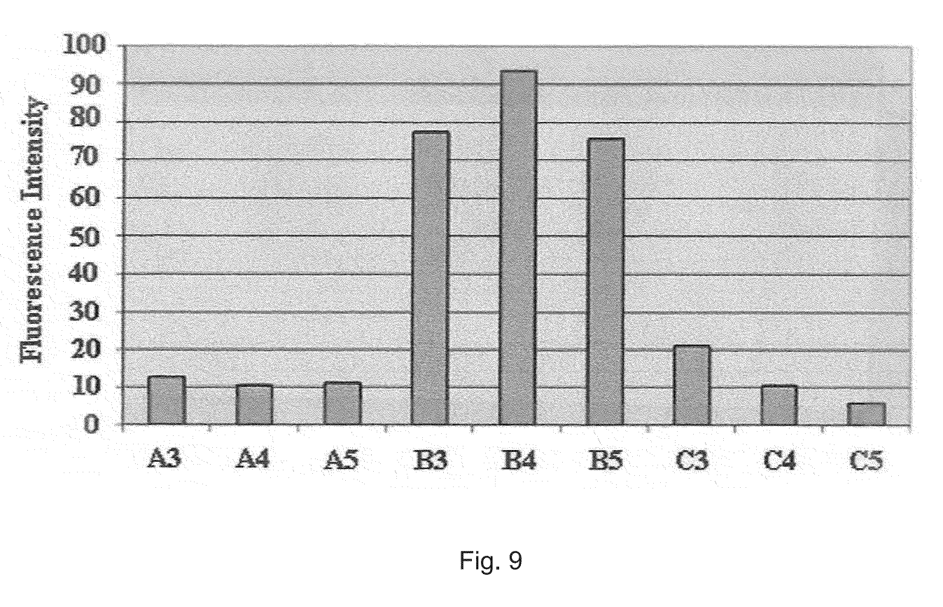

PAA NPs can be targeted to nucleolin with F3-Cys. F3-targeted NPs were prepared using two kinds of F3 peptides: F3 peptide conjugated to NP via one of the 8 lysines available in its sequence and F3-Cys peptide conjugated to NP via cysteine. Cysteine capped NPs served as non-targeted control. Three 25 mg batches of each type of NP contained: 2.6, 5.1 and 7.7 mg F3, (A3-A5) respectively; 2.7, 5.3 and 8 mg F3-Cys (B3-B5) respectively, and 0.29, 0.58 and 0.87 mg Cys (C3-C5) respectively. The fluorescence intensity from PAA NP incubated in vitro with nucleolin positive MDA-MB-435 cells is shown in FIG. 9. The F3-Cys conjugated NPs show considerably higher binding efficiency than non-targeted NPs, while F3 conjugated NPs do not. Conjugation via a cysteine link preserves the specificity of F3 peptide for nucleolin. In addition excess cysteine on the NPs helps to minimize the non-specific binding. Additional experiments (not shown) suggested that the amount of F3-Cys peptide (5.3 mg/25 mg NP) used for B4 NPs was optimal.

Optical properties of post-loaded PAA NPs. The absorption spectrum of PAA NPs post-loaded with both HPPH and cyanine dye (even at 0.5 mg/ml), clearly shows characteristic signatures for both the PS and dye, without aggregation-induced broadening, while the fluorescence spectrum shows strong signals from both components.

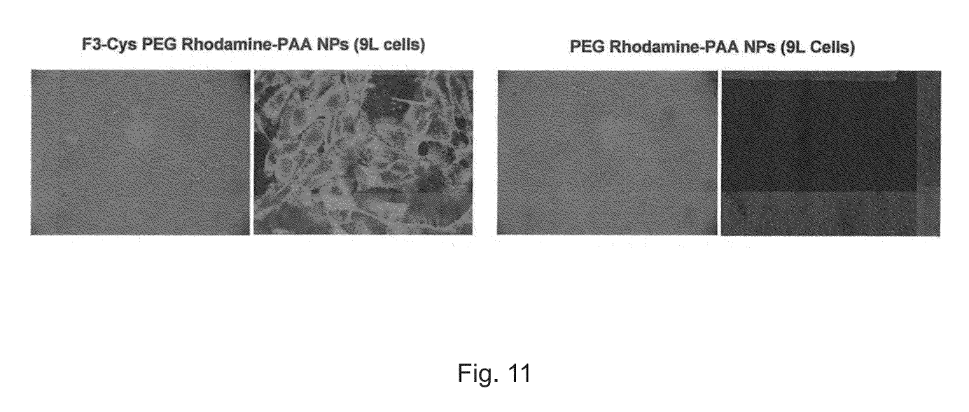

HPPH conjugated PAA NPs with F3-Cys peptide at the outer surface show targeted specificity. F3-mediated specificity is retained in the presence of conjugated HPPH. F3 targeted NPs did targeted NPs did not, indicating that F3-mediated specificity is retained in the presence of conjugated HPPH. F3 targeted NPs did not accumulate in the nucleus. On activation of cells with light at 660 nm only F3-targeted NP caused cell kill (FIG. 11). Cell internalization of F3-targeted NPs was confirmed by fluorescence confocal microscopy.

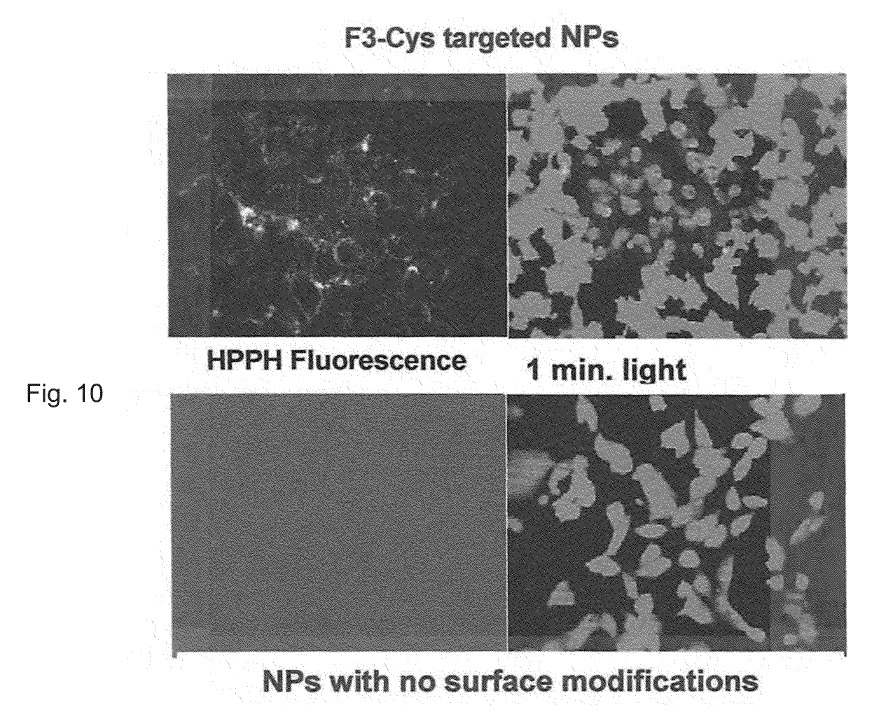

HPPH conjugated PAA NPs with F3-Cyspeptide at the outer surface show targeted specificity. The specificity of targeted NPs was tested by fluorescent imaging (FIG. 10). F3 targeted HPPH conjugated PAA NP specifically bound to MDA-MB-435 cells (expressing nucleolin) while non-targeted NPs did not, indicating that F3-mediated specificity is retained in the presence of conjugated HPPH. F3 targeted NPs did not accumulate in the nucleus. On activation of cells with light at 660 nm only F3-targeted NP caused cell kill (FIG. 11). Cell internalization of F3-targeted NPs was confirmed by fluorescence confocal microscopy.

F3-Cys shows target-specificity in 9L glioma cells. Similar to F3-cys, a pegylated form of F3-Cys PEG on PAA NPs also showed remarkable target-specificity in 9L rat glioma cells which also expresses nucleolin, FIG. 11. (Note: HPPH is replaced with a Rhodamine moiety).

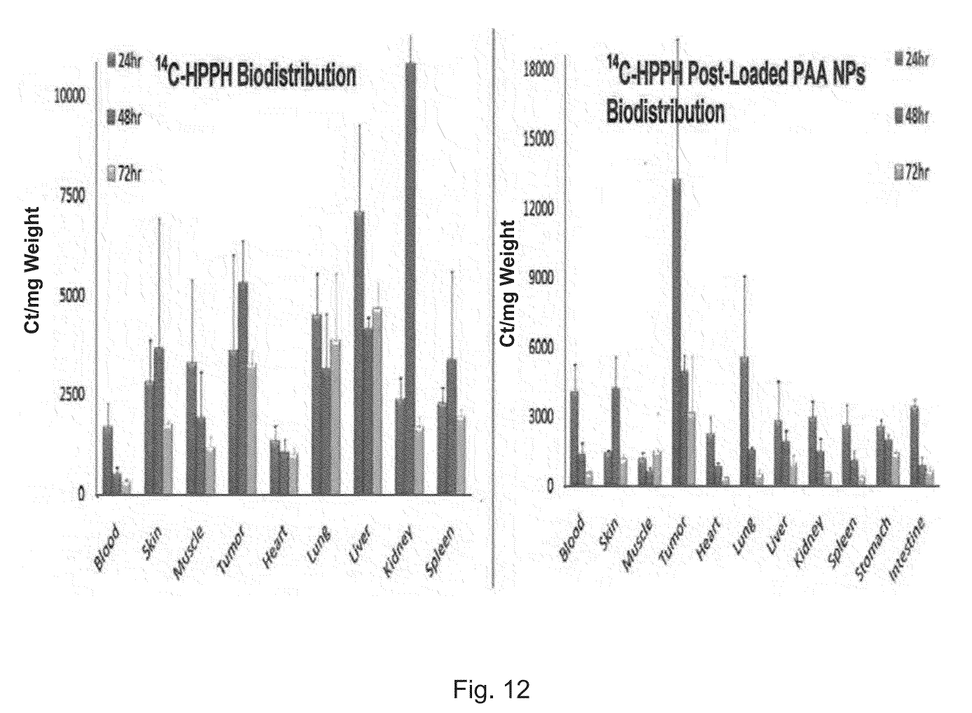

Biodistribution studies: PAA NP Enhances tumor uptake of HPPH. The biodistribution of 14C-HPPH and 14C-HPPH post-loaded PAA NP was performed in BALB/c mice bearing Colon26 tumors at 24, 48 and 72 h post injection (3 mice/time point) and the results are summarized in FIG. 12. As can be seen presence of PAA NPs made a significant increase in tumor uptake with reduced uptake in other organs.

In a preferred embodiment of the invention, a nanoplatform containing a PET/fluorescence imaging photosensitizer derived from chlorophyll-a has significant unexpected advantages. Compared to a free photosensitizer (PS), the corresponding polyacrylamide-based nanoformulation shows a remarkable in vivo enhancement in tumor-imaging and photodynamic therapy. The non-toxic nanoparticles (30-35 nm) formulation drastically change the pharmacokinetic profile of the imaging/therapeutic agent (formulated in 1% Tween 80 and 5%/D5W) with remarkable enhancement in tumor uptake (10% of the injected dose) and reduced uptake in spleen and liver. The labeled (¹²⁴I-) and non-labeled PS in combination show great potential for tumor imaging (PET/fluorescence) and photodynamic therapy in BALB/c mice bearing Colon26 tumors and provides an opportunity for “See and Treat” approach.

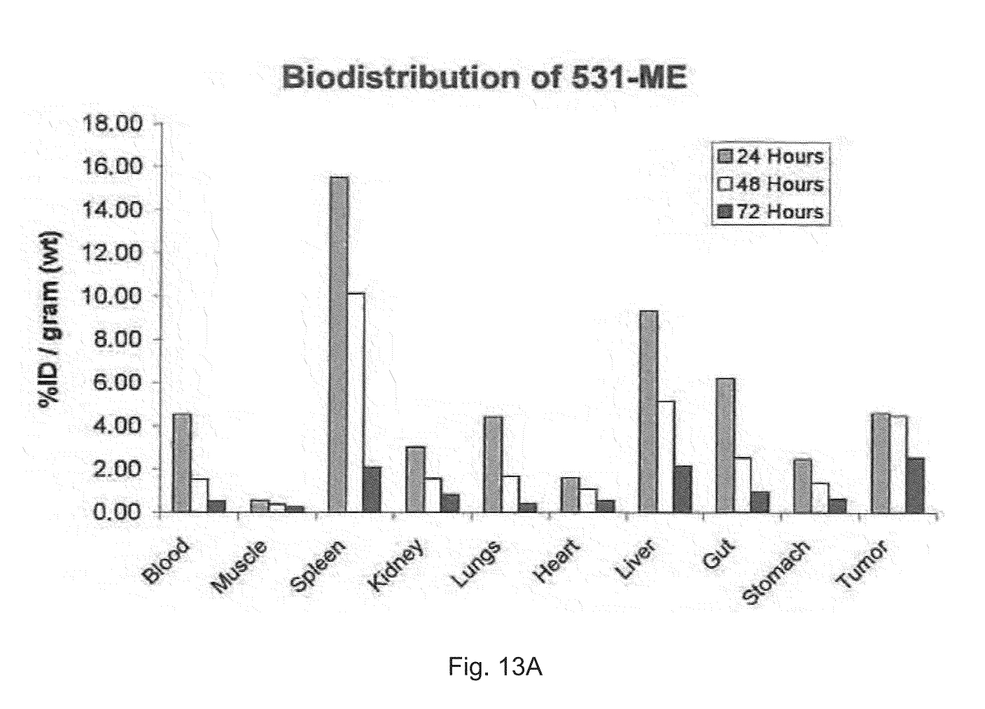

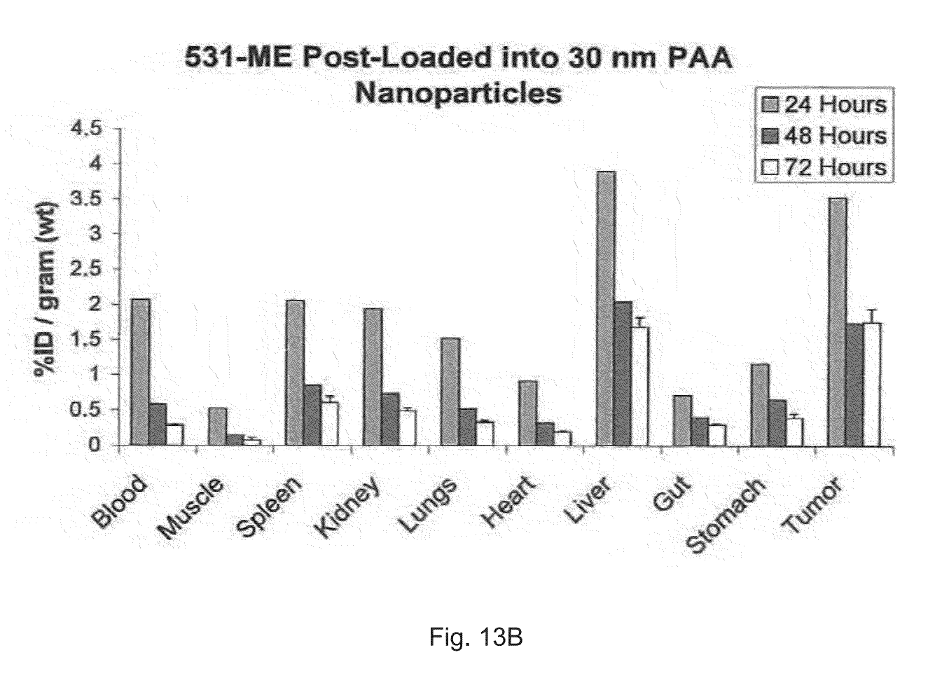

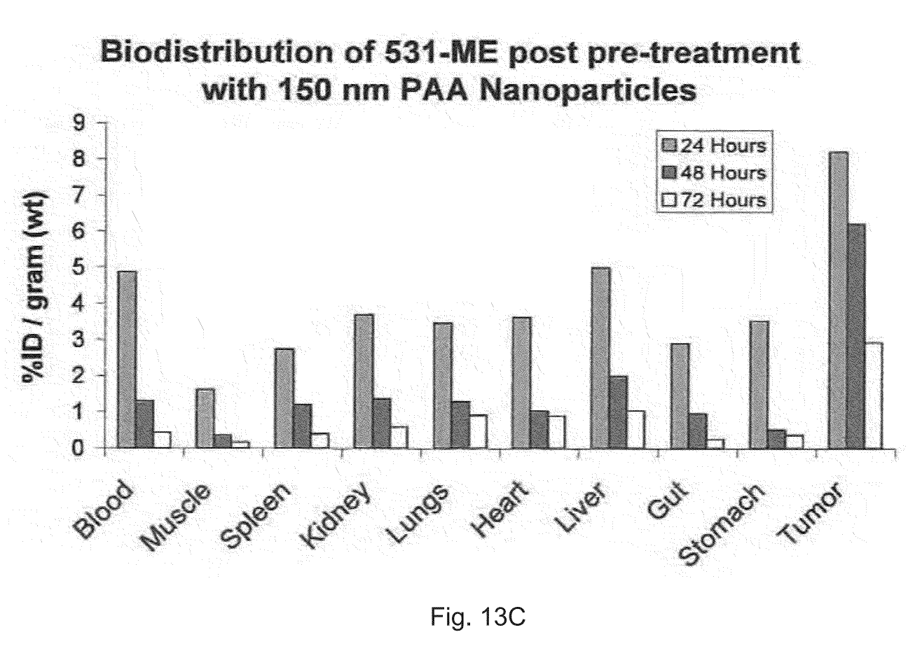

Size of PAA NPs make remarkable difference in tumor-enhancement. The biodistribution of 124I-photosensitizer was investigated using variable sizes of nanoparticles either injecting the NPs first and then administrating the labeled photosensitizer or postloading the labeled photosensitizer to PAA NPs and then perform in vivo biodistribution in mice at 24, 48 and 72 h. The results summarized in FIGS. 13A-13C clearly indicate that the size of PAA NPs makes a significant impact in tumor enhancement. Experiments related to in vivo PDT efficacy of these formulations are currently in progress.

This invention shows the utility of porphyrin-based compounds in a “BIFUNCTIONAL AGENT” for imaging breast tumor and tumor metastasis. Similar to most NPs, PAA NP accumulate in liver and spleen. Their clearance rate from most organs is significantly faster than Ormosil NP and they do not show long-term organ toxicity. Even tumor-avid porphyrin based PS exhibit high uptake in liver and spleen, but are non-toxic until exposed to light. The PS clear from the system quickly (days) without organ toxicity. However, radioactive PS such as the ¹²⁴I-labeled analog 2 (superior to 18F-FDG in PET-imaging of lung, brain, breast and pancreas tumors) with a T1/2 of 4.2 days could cause radiation damage to normal organs. Based on the observation of high uptake of PAA NPs in liver and spleen (below) we postulated that saturating the organs with the non-toxic PAA NPs before injecting the PET agent might reduce uptake and radiation damage by 124I-imaging agent. For proof-of principle blank PAA NPs were first injected (i.v.) into mice bearing Colon26 tumors followed 24 h later by i.v. 124I-analog (100-150 μCi). The mice were imaged at 24, 48 and 72 h post injection and biodistribution studies were performed at each time point summarized in FIG. 8A-8C (only 72 h images shown).

The presence of PAA NPs makes a remarkable difference in tumor contrast with significantly reduced uptake in spleen and liver and improved tumor-uptake/contrast at 24, 48 and 72 h post injection (3 mice/group Similar studies (tumor-imaging and PDT efficacy) in which the labeled PS is post-loaded to variable sizes. Similar studies (tumor-imaging and PDT efficacy) in which the labeled PS is post-loaded to variable sizes PAA NPs are currently in progress.

With the latest advent of small animal micro-PET systems, the resolution of which could reach near 1.2 mm, PET has widened its appeal for research at the drug development stage, as it allows studying the drug distribution in vivo. Most of the porphyrin-based compounds show significantly higher accumulation in the tumor at 24 to 48 h post injection. Therefore for developing multifunctional agents for PET/PDT, we introduced the iodobenzyloxyethyl group at position-3 of the pyropheophorbide-a, which showed tumor-avidity with significant PDT/optical imaging (excitation: 665 nm, emission: 715 nm) efficacy 24 h post-injection. The corresponding ¹²⁴I-analog (half-life 4.2 days) also showed its ability to image various types of tumors in mice models (U87, Colon26, RIF, 4T1, Panc-1), and thus a “Tri-functional Agent” was discovered.

The in vivo biodistribution of the labeled photosensitizer showed significant tumor avidity [4.85% of the injected dose was present in tumors (BALB/c mice bearing Colon26 tumors)], but also produced high uptake in liver and almost 4-fold increase in the spleen. However, the rate of the clearance of the iodinated analog from the spleen, liver and other organs was much faster than tumor at 24, 48 and 72 h post-injection. Most of the photosensitizers derived from chlorophyll-a are nontoxic and PDT being a local treatment modality, the presence of photosensitizer in liver and spleen does not produce any organ toxicity and other side effects. However, in developing radiolabelled agents for tumor-imaging, it becomes necessary to develop an agent/formulation which helps to retain the desired product in tumor for a longer period, but clears off rapidly from the normal organs.

In our present study, we investigated the utility of polyacrylamide (PAA) nanoparticles in delivering the non-labeled and labeled (¹²⁴I-) photosensitizers for PET imaging and PDT. Comparative biodistribution, tumor-targeting ability and normal organ toxicity of the PS and the respective PAA NPs formulation were also investigated. The photosensitizer methyl-3-(1′-m-iodobenzyloxyethyl)pyropheophorbide 1 and the corresponding ¹²⁴I-labeled analog 2 were synthesized by following the methodology developed in our laboratory. To formulate these hydrophobic PS's in an aqueous environment, we post-loaded the PS to biodegradable amine functionalized polyacrylamide nanoparticles (size 25-30 nm) in high concentration. The loading efficiency of the PS was determined by measuring the radioactivity present in the NP formulation after post-loading and after centrifuge filtering the nanoparticles to remove the PS that did not post-load. The activity observed after post-loading was 1.1 mCi and the amount released was 8 μCi which equates to a 99.27% loading efficiency.

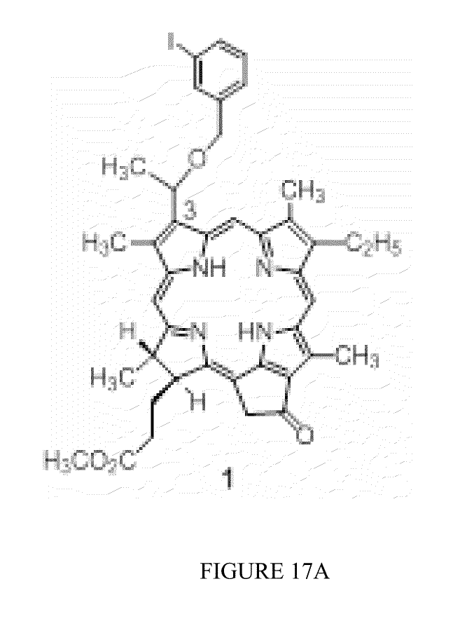

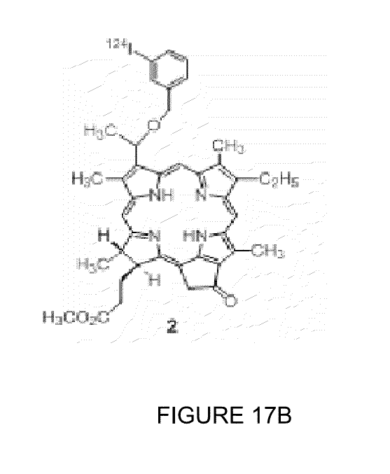

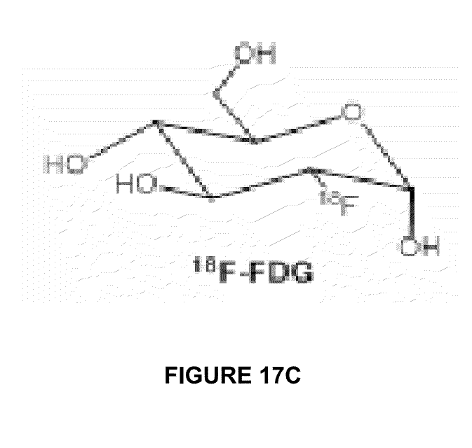

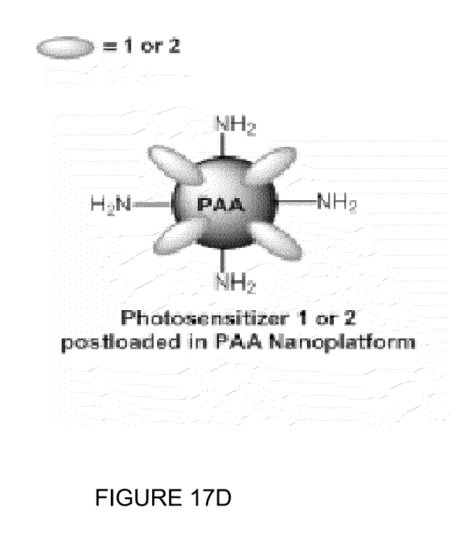

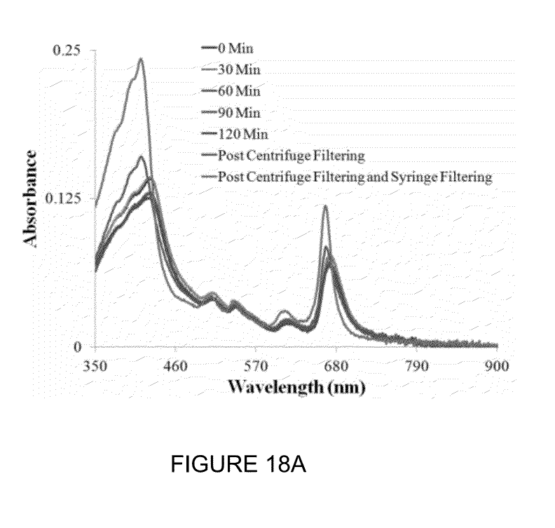

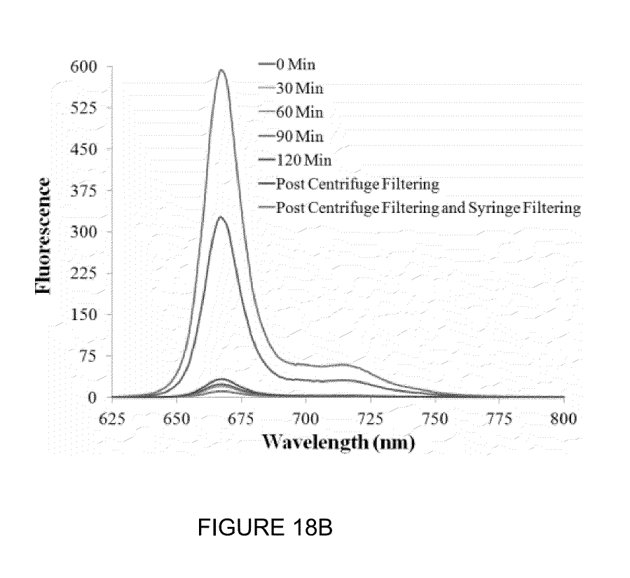

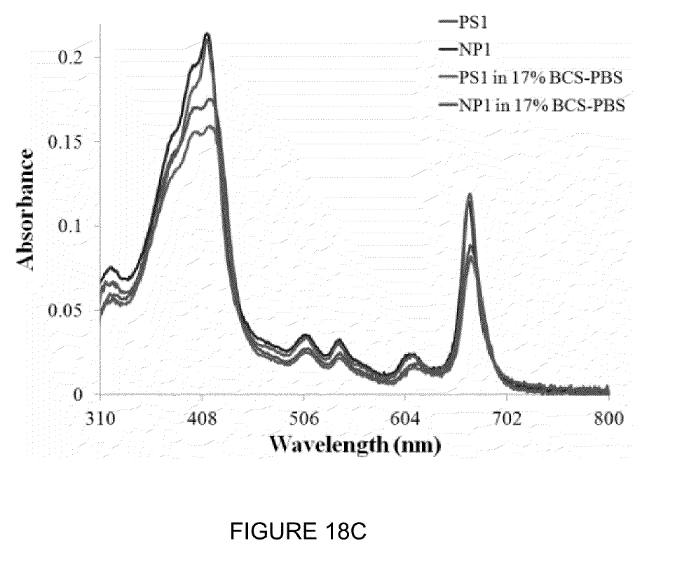

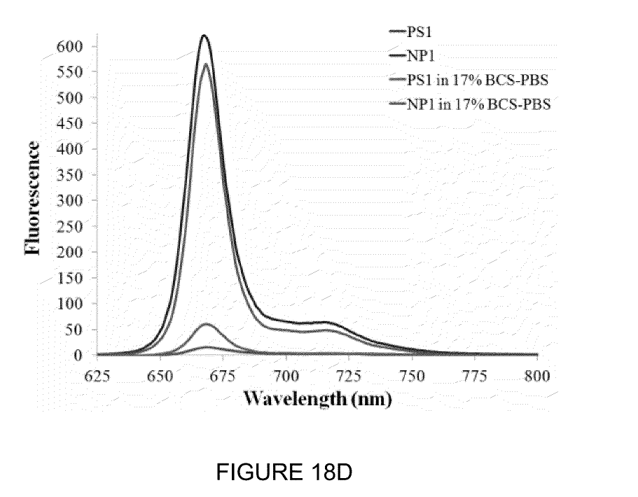

Changes in the absorbance and fluorescence spectra proved to be very informative in assessing the post-loading of PS to the PAA NPs. FIG. 18D shows that at equimolar concentrations, three micromolar, of the non-radiactive photosensiter, FIG. 17A PS1, and the non-radioactive photosensitizer post-loaded to polyacrylamide nanoparticles, NP1, the absorbance value across the whole absorbance spectrum for NP1 is higher. The fluorescence of NP1 was measured and it was found to be 40× more fluorescent than PSI. Because in vivo serum is present, the absorbance and fluorescence spectra were compared when PS1 and NP1 were diluted to equimolar concentrations in 17% Bovine Calf Serum (BCS) in PBS. For the absorbance spectra, the width of the q-band absorbance peak slightly decreased for PS1, whereas for NP1, there was no change.

The slight decrease in the width of the q-band absorbance peak led to a 3.8 fold increase in fluorescence intensity for PS1. For NP1, there was no significant change in the absorbance spectrum when diluted with 17% BCS or PBS and exhibiting similar fluorescence intensity. The aggregation/dis-aggregation properties of PS can be manifested by electronic absorption and fluorescence and were found to be extremely useful in monitoring the post-loading of the PS in PAA NPs. The PS dissolved in aqueous DMSO solution before post-loading to NPs was in a highly aggregated form and produced weak fluorescence and broad absorption. However, on stirring the solution with the NPs, the PS started disaggregating with its simultaneous increase in post-loading to the NPs; during the time of post-loading there was an increase in the fluorescence intensity. No further change in electronic absorption of NP1 was observed after 2 h of magnetic stirring. Upon centrifuge and syringe filtering the nanoparticle solution, the width of the absorbance peak further decreased with a significant increase (18-fold) in fluorescence intensity. The sharpening of the absorbance peak clearly indicated that the PS in post-loaded form was in a less aggregated state. The increase in the PS's fluorescence from the start to the end of post-loading in PAA NPs was >50-fold. These findings are of immense interest as it provides a simple approach for formulating hydrophobic photosensitizer(s) while retaining their photophysical properties.

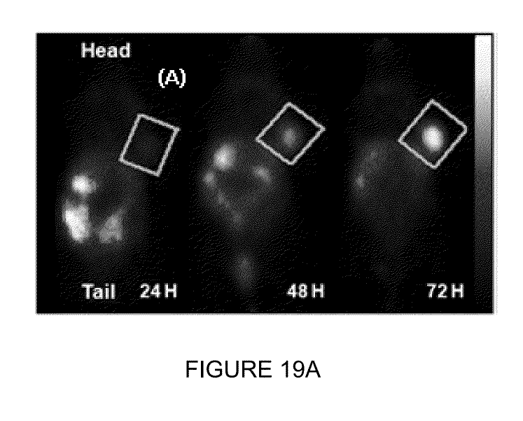

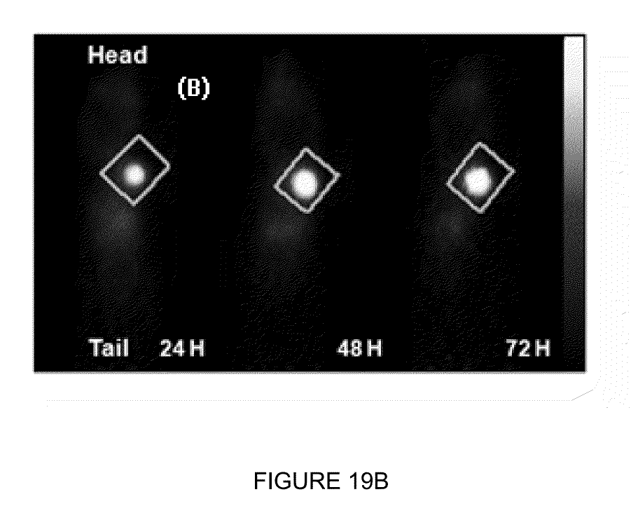

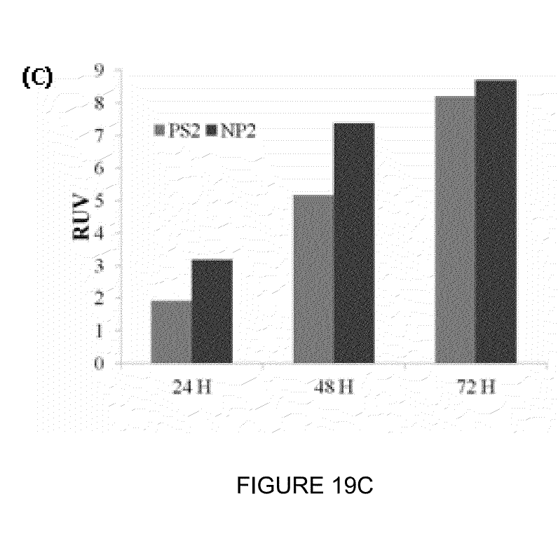

Our next step was to compare the in vivo PET images of the ¹²⁴I-PS2 with the corresponding PAA nanoparticles, NP2. From the results summarized in FIG. 19C, it can clearly be seen that with the post-loaded formulation, the tumor can be demarcated easily at 24, 48 and 72 h post-injection (i.v.) with reduced background signal. The relative uptake value (RUV) of the imaging agent to quantify the visibility of the tumor was calculated by the following formula:

$$RUV = \frac{max\_voxel \_activity \_concentration \_in \_tumor ( Bq / cc )}{\frac{activity\_in \_the \_imaged \_body ( Bq )}{volume\_of \_the \_imaged \_body ( cc )}} .$$

An iso-contour ROI was used to define the body volume for the mouse. The lower threshold was set to about 2-4% of the maximum voxel intensity and adjusted according to visual inspection. The RUV calculation correlates with the established SUV and allows for the measurement of the relative tumor uptake without the need for measuring excreted radioactivity from the time of injection to the scan time. FIG. 19C shows that the RUV for both PS2 and NP2, which increased over time, i.e., the visibility of the tumor compared to the background signal increases. For NP2, the RUV value was consistently higher starting at 3.17 (24 h post-injection) and optimizing at 8.7 (72 h post-injection), whereas for the PS2 the RUV increases with time from 2 to 8.2 72 h post-injection. At 24 h post-injection, the RUV of NP2 and PS2 is higher than the RUV of PS2 by 67%. Both PET images and the RUV data clearly show that the post-loading approach enhanced the detection of Colon26 tumors.

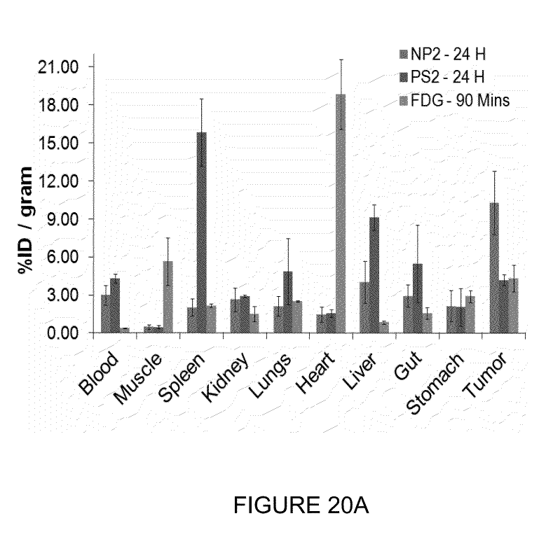

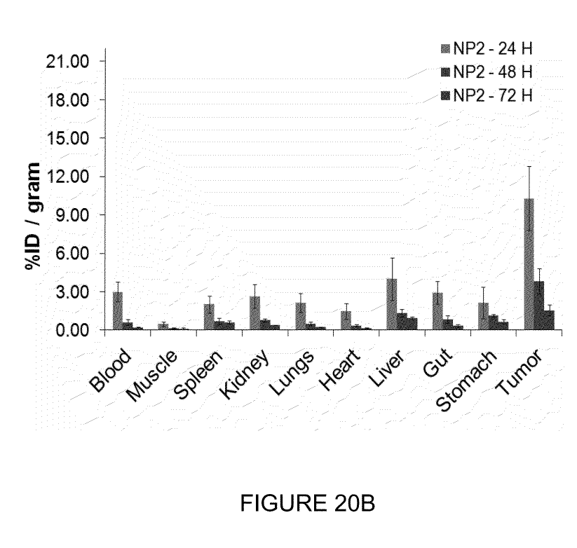

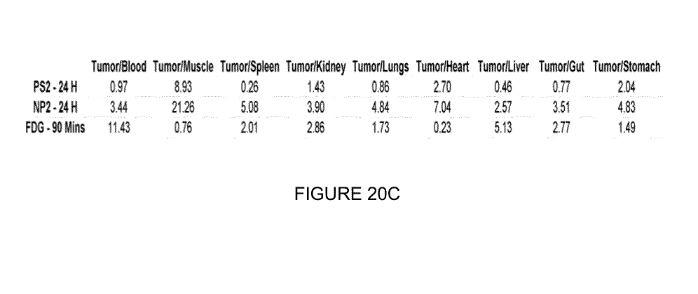

For investigating the superiority of the labeled PS over ¹⁸F-FDG, a comparative biodistribution study of PS2, NP2 and ¹⁸F-FDG was performed in BALB/c mice bearing Colon26 tumors. From the results summarized in FIG. 20A it can be seen that by post-loading PS2 to PAA NPs, the percentage injected dose/gram (% ID/gram) in the tumor at 24 h post-injection significantly increased from an average value of 4.61% to 10.28% (P=0.008). The % ID/g for ¹⁸F-FDG in the same tumor model was also compared against NP2 and a significant difference (4.31% versus 10.28%, P=0.019) was observed. This reflects a 223% increase in PS2 (NP formulation) present in the tumor as compared to free PS2 (without NP formulation) and interestingly an increase of 239% if compared against ¹⁸F-FDG. Compared to PS2, the NP2 formulation showed a remarkable decrease in accumulation in the spleen and liver and significantly less in the heart and muscle if compared to ¹⁸F-FDG alone. The % ID/gram of PS2 present in the spleen and liver at 24 h post-injection was 15.51 and 9.32%, respectively. With nanoparticle formulation NP2, the amount decreased to 2.02 and 3.99%, respectively.

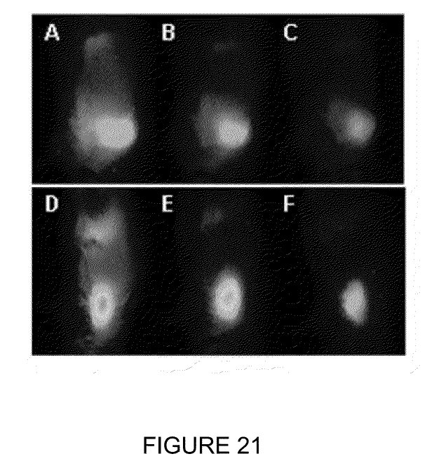

In vitro release kinetics study of non-labeled PS1 from the corresponding nanoformulation NP1 was performed in 1% human serum albumin (HSA) immediately after adding human serum albumin (HSA), 2, 4, and 24 h post addition of NP1 to a 1% HSA solution. The retention over time was >95% (see supplemental information). To further confirm that the PS post-loaded nanoformulation accumulated more in the tumor than the PS alone, whole body fluorescence reflectance imaging was performed of PS1 and NP1. The results summarized in FIG. 21 confirm that the tumor-uptake of the PS is higher in the NPs formulation than it is formulated without NPs.

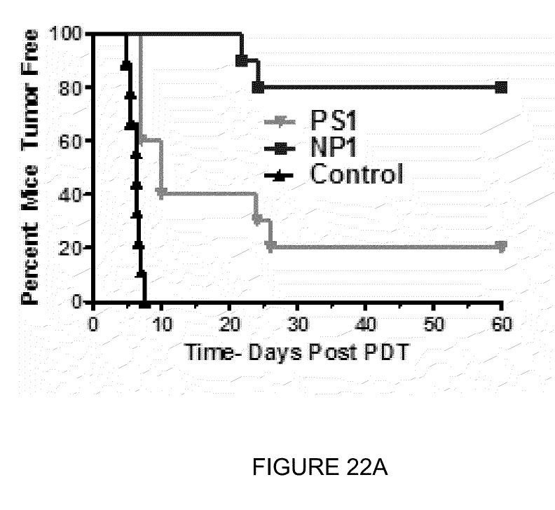

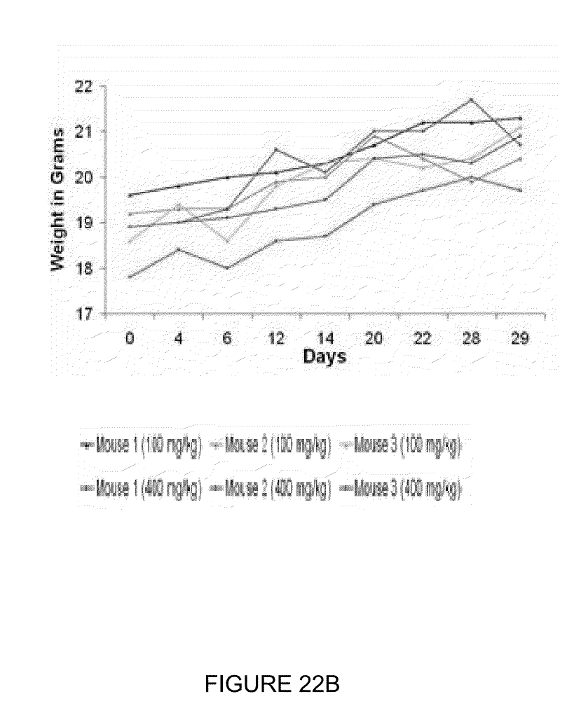

We also investigated the organ toxicity of the PAA nanoparticles at four doses: 100 mg/kg (required for post-loading the ¹²⁴I-PS at the imaging dose); 200 mg/kg (2-fold higher than the required dose); 300 mg/kg (3-fold higher than the required dose); and 400 mg/kg (4-fold higher than the required dose). At all three doses the mice (3 BALB/c mice/group) were monitored for weight loss/gain and other signs of distress for 30 days post-injection of the nanoparticles. At day 30, the mice were necropsied and the organs were analyzed for toxicity by H&E staining. No acute toxicity was observed even at 4-fold higher than the imaging dose. The H&E staining for the liver, spleen, heart, kidney and lung (400 mg/kg group) along with the corresponding controls (BALB/c mice void of nanoparticles), and the weight of the mice for the duration of the study (30 days) are summarized in FIG. 22A. To investigate the impact of nanoparticle formulation in PDT efficacy, PS1 and the corresponding nanoparticles formulation NP1 were evaluated for in vivo PDT efficacy under similar treatment parameters. In brief, BALB/c mice bearing Colon26 tumors (10 mice/group) were injected with PS or the NP formulation at a dose of 1.0 μmole/kg. The tumors were exposed to light at 665 nm (dose: 135 J/cm², 75 mW/cm²) at 24 h post injection and the tumor response was recorded daily following the animal protocol approved by the institutional IACUC committee. The percentages of tumor cure are shown in FIG. 22A. The Kaplan-Meier survival graph highlights the remarkable enhancement of long-term tumor cure with NP formulation, from 20% (2/10 mice were tumor-free with the PS alone) to 80% (8/10 mice were tumor free with NPs-PS formulation).

Further experimental details are set forth below.

Synthesis of Blank AFPAA Nanoparticles: To a dry 100 mL round bottom flask add 45 mL of hexane (VWR, USA) and stir for 45 min-1 h under a constant purge of argon. AOT (1.6 g, Sigma-Aldrich, USA) and Brij 30 (3.1 g or 3.3 mL, Sigma-Aldrich, USA) was added to the reaction flask and stirred under argon protection for 20 min. Acrylamide (711 mg, Sigma-Aldrich, USA), APMA (89 mg, Polysciences, USA) and biodegradable AHM (428 mg or 375 L, Sigma-Aldrich, USA) were dissolved in phosphate buffered saline (2 mL) (PBS, 10 mM pH=7.4) and the entire mixture was sonicated (5 min) to obtain a uniform solution. This solution was then added to the hexane reaction mixture and vigorously stirred for 20 min at room temperature. The polymerization of acrylamide was initiated by adding 40 L of freshly prepared aqueous ammonium persulfate solution (10% w/v, Sigma-Aldrich, USA) and TEMED (40 L, Sigma-Aldrich, USA). The resulting solution was stirred vigorously overnight. At the completion of polymerization, hexane was removed by rotary evaporation and the particles were precipitated by addition of ethanol (50 mL). The surfactant and residual monomers were washed away from the particles with ethanol (150 mL, Pharmaco-Aaper, USA) followed by washing with water (100 mL) five times each in an Amicon ultra-filtration cell equipped with a Biomax 300 kDa cutoff membrane (Millipore, USA). The concentrated nanoparticles were lyophilized for two days, and stored in the freezer. The nanoparticles were reconstituted by suspending in PBS. Once in liquid form, the nanoparticles are stored at 4° C.

Acronyms: AFPAA (Amine Functionalized Polyacrylamide Nanoparticles), Dioctyl Sulfosuccinate Sodium Salt (AOT), 3-(aminopropyl)methacrylamide (APMA), 3-(acryloyloxy)-2-hydroxypropyl methacrylate (AHM), and Phosphate Buffered Saline (PBS).

Post-Loading of the PS1 to Blank AFPAA Nanoparticles: The lyophilized AFPAA NPs are dissolved in 1% Tween-80/PBS (pH 7.4, 10 mM) to a final concentration of mg/1 mL. The NPs are sized by DLS prior to the post-loading of PS1 to ensure that they are of the appropriate size. PS1 is dissolved in DMSO to a final concentration of 20 mM. 20 μL of PS1 in DMSO is added to 2 ml of NP solution and is magnetically stirred at a constant rpm for a minimum of 2 hours. The NP solution is transferred to an Amicon Ultra-4 30 kDa centrifuge filter and centrifuged at 4,000 rpm for 40 minutes to remove excess DMSO, Tween-80, and PS1 that did not post-load. The filtrate is spectrophotometrically measured and if signal for PS1 is detected, the retentate is reconstituted to the original volume with PBS and recentrifuged. This is continued until no signal is detectable in the filtrate spectrophotometrically. The nanoparticle solution is syringed filtered and then the concentration of PS1 is measured in ethanol using the Beer's-Lambert Law (molar extinction coefficient: 47,500 L m⁻¹ cm⁻¹). The nanoparticles may cause scattering in the absorbance spectra. If this occurs, the nanoparticle solution can be centrifuge filtered in a microfuge membrane-filter (NANOSEP 100K OMEGA, Pall Corporation) at 14,000 RPM for 10 minutes. The filtrate is used to calculated the concentration of PS1 that was post-loaded to the PAA NPs. The nanoparticles are syringe filtered with a 0.2 μm syringe filter and stored at 4° C. for further use.

Post-Loading of the PS2 to Blank AFPAA Nanoparticles: The lyophilized AFPAA NPs are dissolved in 1% Tween-80/PBS (pH 7.4, 10 mM) to a final concentration of 10 mg/1 mL. The NPs are sized by DLS prior to the post-loading of ¹²⁴I-labeled PS2 to ensure that they are of the appropriate size. 2.1 mL of the NP solution is added to the vial containing PS2 dissolved in a 100 μL of DMSO. The solution is magnetically stirred at a constant rpm for a minimum of 2 hours. The NP solution is transferred to an Amicon Ultra-4 30 kDa centrifuge filter to remove excess DMSO, Tween-80, and PS2 that did not post-load. 1.3 mL of additional PBS is used in the transfer process to ensure that the entire radioactivity is transferred from the vial to the centrifuge filter. The NP solution was transferred to the centrifuge filter and was centrifuged at 4,000 rpm for 40 min. Post-centrifuge filtration, the amount of radioactivity released from the NP is measured. If the activity in the filtrate is greater than 5%, then the retentate is reconstituted to the original volume and recentrifuged. This process is repeated until less than 5% of the total radioactivity is found in the filtrate. The rententate is reconstituted to 1.5 mL with PBS to ensure that each 100 μL of NP solution will contain ˜60 μCi of activity.

Release Kinetic Studies. In brief, the nanoparticles post-loaded PS1 were mixed with 1% aqueous Human Serum Albumin, HSA, solution (w/v). The absorbance is measured of the solution and is marked as the stock absorbance. The solution is then centrifuged in an Amicon Ultracel-4, 100 kDa centrifuge filter at 4,000 RPM for 30 minutes. The filtrate is marked as filtrate #1 and is measured spectrophotometrically. The retentate is reconstituted to the original volume with 1% HSA, thoroughly mixed with a pippet, and re-centrifuge filtered. The filtrate is marked filtrate #2 and is measured spectrophotometrically. To measure what is retained by the NP after the two wash steps, the NPs are reconstituted with 1% HSA, thoroughly mixed with a pippet and is measured spectrophotometrically.

Characterization of the Size of the PAA Nanoparticle Formulation Used for Photodynamic Therapy/Optical Imaging and Toxicology Studies. The hydrodynamic diameter of the blank nanoparticle and NP1 were measured using the Nicomp 370 Submicron Particle Analyzer (Nicomp, Santa Barbara, Calif.). The NPs were diluted in a borosilicate glass tube with PBS (10 mM, pH 7.4) to achieve an intensity count of 300 kHz. The samples were measured in triplicate with each run lasting five minutes. The volume intensity weighting was used when determining the mean hydrodynamic diameter.

Measuring the Concentration of PS1 in NP1 and PS1: To calculate the concentration of PS1 within NP1, NP1 is diluted in ethanol and measured spectrophotometrically using a Varian (Cary-50 Bio) with an extinction coefficient of 47,500

$$( \frac{L}{moles * cm} ) .$$

To remove the scattering in the absorbance spectra, the nanoparticles were centrifuged filtered with a Microfuge membrane-filter (NANOSEP 100K OMEGA, Pall Corporation) at 5,000 rpm for 10 minutes. The NPs are retained above and the PS loaded within the NP is in the filtrate. The filtrate is measured spectrophotometrically according to the Beers-Lambert law. To calculate the concentration of PS1, PS1 is diluted in methanol and measured spectrophotometrically as described above with the same extinction coefficient.

Comparative Absorbance and Fluorescence Spectra Measurements of PS1 and NP1: The absorbance spectra was collected from 350-900 nm with the concentration of PS1 and NP1 in either PBS or 17% BCS-PBS was three micromolar. The fluorescence measurements were recorded using a Cary Eclipse fluoremeter (Varian Inc, USA). The excitation wavelength for PS1 diluted in PBS, PS1 diluted in 17% BCS, NP1, and NP1 diluted in 17% BCS was excited 413, 416, 413, 413 nm, respectively. The fluorescence emission was collected from 600-800 nm. For both formulations the excitation and emission slit was set to 5 nm and the PMT voltage was set to medium.

The absorbance and fluorescence spectra was measured at various times throughout the post-loading procedure. The times points included, before magnetic stirring (0 min), after magnetic stirring: 30 min, 60 min, 90 min, and 120 min, after centrifuge filtration, and after syringe filtering NP1 with a 0.2 μm cellulose acetate syringe filter. The absorbance spectra was collected from 300-800 nm with the concentration of each sample equaling three micromolar. From time 0 to time 120 minutes post magnetic stirring, the excitation wavelength was 425 nm and the excitation wavelength for the nanoparticle sample that was centrifuge filtered, and syringe filtered was 414, and 413 nm, respectively.

Synthesis of PS2: The trimethyltin analogue of PS2 (50 μg) was dissolved in 50 μL of 5% acetic acid in methanol, and 100 μL of 5% acetic acid in methanol was added to Na¹²⁴I in 10 μL of 0.1 N NaOH. The two solutions were mixed and 10 μL of N-Chlorosuccinimide in methanol (1 mg/mL) was added. The reaction mixture was incubated at room temperature for 8 min and the reaction product was injected on a HPLC column (Phenomenex Maxsil C8 5 μm), which was eluted with a 90:10 mixture of methanol and water at a flow rate of 1 mL/min. The labeled product was collected. After the product was dried, it was then dissolved in 10% ethanol in saline for injection into mice. For use in forming NP2, the dried product was dissolved in 100 μL of DMSO.

Comparative PET Imaging: BALB/c mice were imaged using the microPET FOCUS 120, a dedicated 3D small-animal PET scanner (Concorde Microsystems Incorporated) at the State University of New York at Buffalo under the Institutional Animal Care and Use Committee (IACUC) guidelines. 9-10 BALB/c mice were subcutaneously injected with 1×10⁶ Colo-26 cells in 50 μL of RPMI-1640 media in the axilla region, and the tumors were grown until they reached 6 mm in diameter (approximately 7 days). 55-181 μCi of PS 2 and/or nanoconstruct 2 were injected and imaged 24, 48, and 72 H post injection. The mice were imaged head prone under a protocol set for 30 minutes. Throughout the acquisition of the images, the mice were continuously anesthetized by inhalation of isoflurane.

Radioiodine uptake by the thyroid or stomach was not blocked. All mice that were imaged were marked with a cross-line on their back to provide a reference landmark for consistently positioning them in a similar position each day they were imaged. The acquired data were rebinned with FORE algorithm 20 and reconstructed using the 2D OSEM algorithm. The dead-time and singles-based random coincidence corrections were applied to all the PET studies. The RUV results were calculated from PET images with attenuation and scatter corrections.

Biodistribution Studies. γ Well Counter. All studies were performed as per IACUC guidelines. Nine mice were injected i.v. with 30-90 μCi of PS2 or NP2. Three mice were sacrificed at each of the following time points, 24, 48, and 72 h post-injection and the blood and body organs, tumor, heart, liver, spleen, kidney, lung, muscle, gut, and stomach were removed. After all the fluids and tissues/organs were weighed, the amount of radioactivity in each sample was measured by a γ well counter. The radioactivity present in each sample was calculated as a percentage of the injected dose per gram of the tissue (% ID/g). A statistical analysis (standard deviation and the unpaired, two-tailed student t-test) was performed using Microsoft Excel to assess if the difference in radioactive uptake of PS2 and NP2 was significantly different (P value <0.05).

Whole-Body Fluorescence Reflectance Imaging: Three BALB/c mice bearing subcutaneous Colon-26 tumors were either injected i.v. with 1.0 mole/kg of PS1 or NP1. 24, 48, and 72 hours post-injection, the mice were imaged with a 12-bit CRI Nuance camera in the mono mode. PS1 and NP1 were excited with an argon pumped dye laser at 665 nm and the fluorescence was collected with a 695 and 700 nm long pass filter.

Optical Imaging Analysis: The whole-body fluorescence reflectance images were analyzed in ImageJ (NIH, USA). All the images were set to the same lookup table and brightness values.

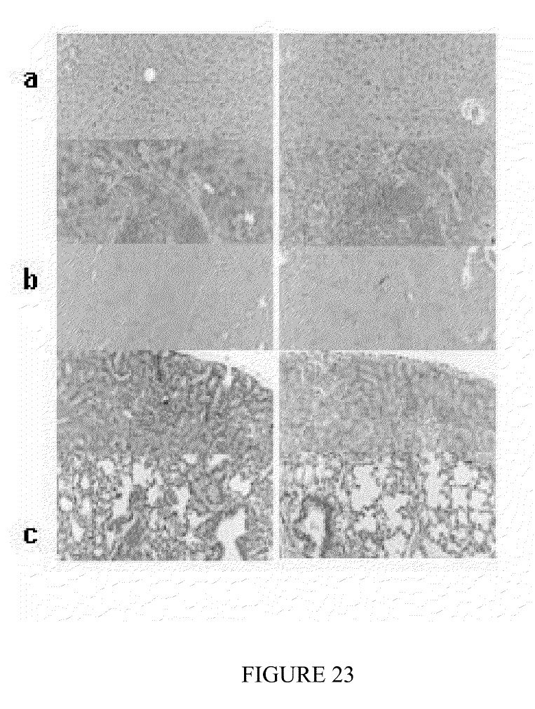

Toxicological Studies: Three female BALB/c mice (Jackson Laboratory, Bar Harbor, Me.) were injected intravenously with 100, 200, 300, and 400 mg/kg of polyacrylamide nanoparticles with mice receiving 100 mg/kg per day. Over a thirty-day period, the weight and behavioral changes were monitored. Day 30, the mice were sacrificed, placed in 10% formalin, and the following organs were analyzed by conventional H.E. staining: trachea, esophagus, urinary bladder, diaphragm, colon, jejunum, duodenum, pancreas, lung (right and left), liver, spleen, thymus, heart, ovary (right and left), uterus, kidney, skin, brain, and bone marrow of the sternum. FIG. 23 shows the representative stained images for the control and 400 mg/kg injected group for the liver, spleen, heart, kidney, and lung at 200× magnification. Dr. Karoly Toth performed the histopathological analysis at Roswell Park Cancer Institute.

In-vivo Photosensitizing Efficacy: 20 BALB/c mice (Jackson Laboratory, Bar Harbor, Me.) were subcutaneously injected with 1×10⁶ Colon 26 cells in RPMI 1640 media (axilla region). The tumors were grown to 4-5 mm in diameter prior to the laser light treatment. The mice were separated into two groups of 10. In the first group (n=10), 1 μmole/kg of PS1 diluted in D5W or dextrose 5% in water was injected intravenously 24 hours prior to PDT. The day before the light treatment, the hair in the treatment area is depilated with the depilatory cream, Nair. In the second group (n=10), NP1 (1 μmole/kg), was injected intravenously 24 hours prior to the light treatment. For the laser treatment, the argon-pumped dye laser was set to 665 nm with a monochromator, and the fluence and fluence rate used was 135 J/cm² and 75 mW/cm². After treatment, the mice were observed for, necrotic scabbing, weight loss, and tumor regrowth. Tumor regrowth is calibrated by two orthogonal measurements, length and width and the tumor volume is calculated according to the formula

$$( \frac{L * {W}^{2}}{2} ) .$$

If the tumor regrowth reached a volume of 400 mm³, the mice were euthanized according the institute policy.

Statistical Analysis: The error bars used in the biodistribution studies are represented as the mean±S.D. To assess for significance, the two-tailed student's t-test was performed with P<0.05 being significant. For the release kinetics data, the data is plotted as the mean±S.E. The error bars and the statistical test was performed in Microsoft Excel. For the in vivo PDT efficacy studies, the statistical test used is the Mantel-Cox test.

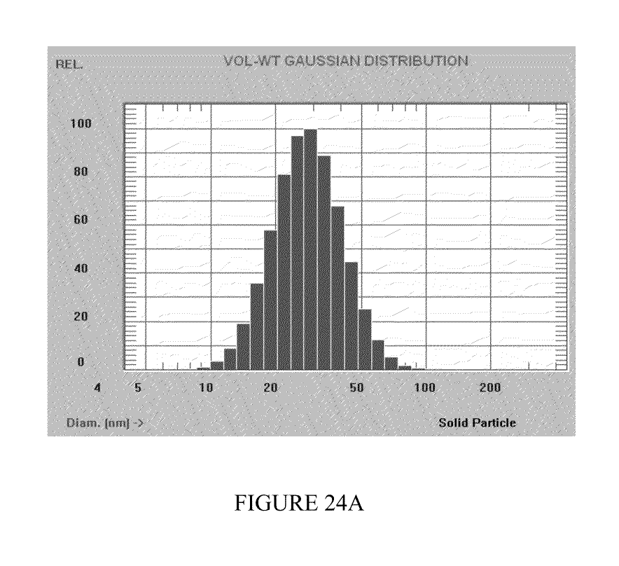

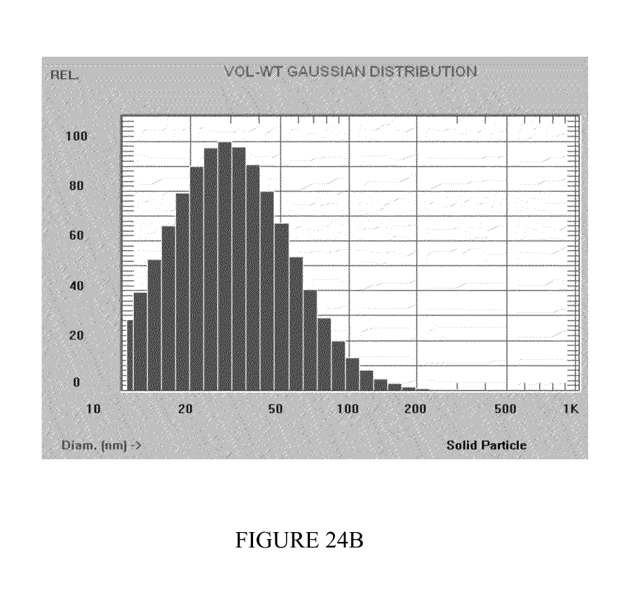

FIG. 24A shows the DLS for Blank PAA NPs used for the toxicological studies and FIG. 24B shows the DLS for NP1 in Tween-80/PBS (concentration of Tween-80 is <1%). The mean diameter is 30 nm, and 35.1 nm for FIGS. 24A and 24B, respectively.

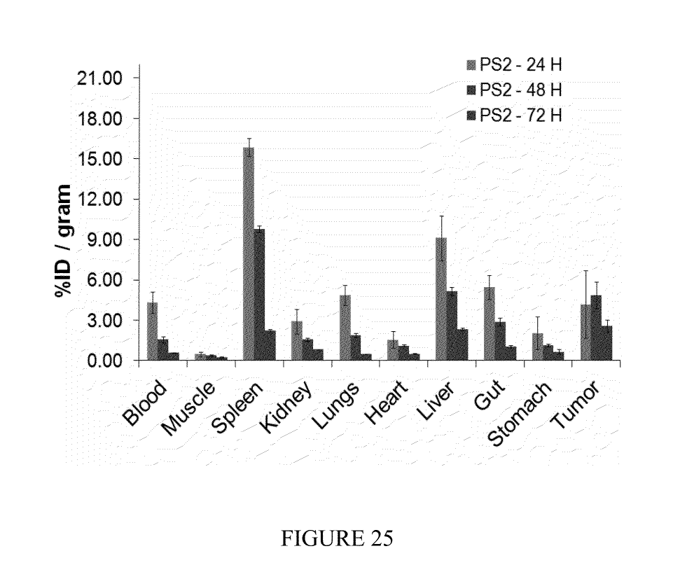

FIG. 25 shows in vivo biodistribution of PS2 24, 48, and 72 hours post tail vein injection in BALB/c (3 mice/group) mice bearing subcutaneous Colon26 tumors on the right shoulders.

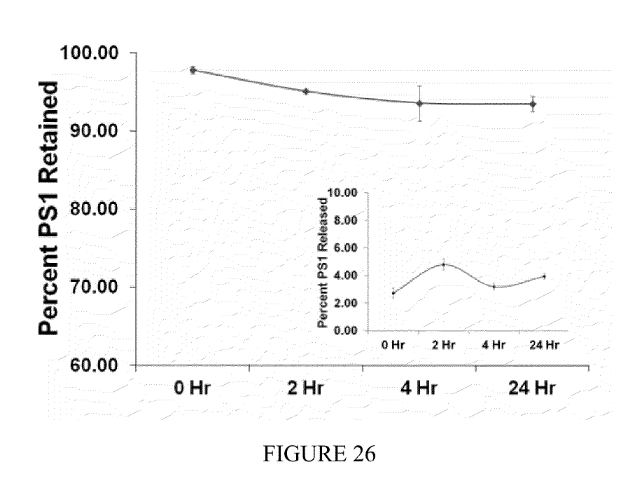

FIG. 26 shows Release/Retention Profiles of PS1 from NP1 in a 1% Human Serum Albumin (HSA) solution. The release/retention was measured immediately upon addition NP1 in 1% HSA (0 Hr), 4, and 24 Hr post-addition of NP1 in a 1% HSA solution. Each experiment was done in triplicate with each time point being the mean. The error bars are standard error of the mean.

What is claimed is:

1. A composition comprising PAA nanoparticles containing at least one tetrapyrollic photosensitizer and a PET imaging agent.

2. The composition of claim 1 wherein at least one photosensitizer comprises a moiety containing ¹²⁴I and also acts as an imaging agent.

3. The composition of claim 1 wherein the tetrapyrollic photosensitizer has the structural formula: 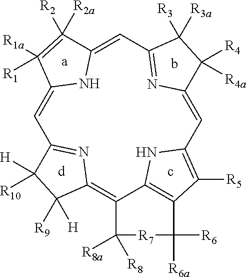
or a pharmaceutically acceptable derivative thereof, wherein:
R₁ and R₂ are each independently substituted or unsubstituted alkyl, substituted or unsubstituted alkenyl, —C(O)Rₐ or —COORₐ or —CH(CH₃)(ORₐ) or —CH(CH₃)(O(CH₂)ₙXRₐ) where Rₐ is hydrogen, substituted or unsubstituted alkyl, substituted or unsubstituted alkenyl, substituted or unsubstituted alkynyl, or substituted or unsubstituted cycloalkyl; where R₂ may be —CH═CH₂, —CH(OR₂₀)CH₃, —C(O)Me, —C(═NR₂₁)CH₃ or —CH(NHR₂₁)CH₃
where X is an aryl or heteroaryl group;
n is an integer of 0 to 6;
where R₂₀ is methyl, butyl, heptyl, docecyl or 3,5-bis(trifluoromethyl)-benzyl; and
R₂₁ is 3,5-bis(trifluoromethyl)benzyl;
R₁ₐ and R₂ₐ are each independently hydrogen or substituted or unsubstituted alkyl, or together form a covalent bond;
R₃ and R₄ are each independently hydrogen or substituted or unsubstituted alkyl;
R₃ₐ and R₄ₐ are each independently hydrogen or substituted or unsubstituted alkyl, or together form a covalent bond;
R₅ is hydrogen or substituted or unsubstituted alkyl;
R₆ and R₆ₐ are each independently hydrogen or substituted or unsubstituted alkyl, or together form ═O;
R₇ is a covalent bond, alkylene, azaalkyl, or azaaraalkyl or ═NR₂₀ where R₂₀ is 3,5-bis(tri-fluoromethyl)benzyl or —CH₂X—R¹ or —YR¹ where Y is an aryl or heteroaryl group;
R₈ and R₈ₐ are each independently hydrogen or substituted or unsubstituted alkyl or together form ═O;
R₉ and R₁₀ are each independently hydrogen, or substituted or unsubstituted alkyl and R₉ may be —CH₂CH₂COOR² where R² is an alkyl group that may optionally substituted with one or more fluorine atoms;
each of R₁-R₁₀, when substituted, is substituted with one or more substituents each independently selected from Q, where Q is alkyl, haloalkyl, halo, photosensitizereudohalo, or —COOR_{b} where R_{b} is hydrogen, alkyl, alkenyl, alkynyl, cycloalkyl, aryl, heteroaryl, araalkyl, or OR_{c} where R_{c} is hydrogen, alkyl, alkenyl, alkynyl, cycloalkyl, or aryl or CONR_{d}Rₑ where R_{d} and Rₑ are each independently hydrogen, alkyl, alkenyl, alkynyl, cycloalkyl, or aryl, or NR_{f}R_{g} where R_{f} and R_{g} are each independently hydrogen, alkyl, alkenyl, alkynyl, cycloalkyl, or aryl, or ═NRₕ where Rₕ is hydrogen, alkyl, alkenyl, alkynyl, cycloalkyl, or aryl, or is an amino acid residue and at least one of R₁-R₁₀ is substituted with ¹²⁴I;
each Q is independently unsubstituted or is substituted with one or more substituents each independently selected from Q₁, where Q₁ is alkyl, haloalkyl, halo, photosensitizereudohalo, or —COOR_{b} where R_{b} is hydrogen, alkyl, alkenyl, alkynyl, cycloalkyl, aryl, heteroaryl, araalkyl, or OR_{c} where R_{c} is hydrogen, alkyl, alkenyl, alkynyl, cycloalkyl, or aryl or CONR_{d}Rₑ where R_{d} and Rₑ are each independently hydrogen, alkyl, alkenyl, alkynyl, cycloalkyl, or aryl, or NR_{f}R_{g} where R_{f} and R_{g} are each independently hydrogen, alkyl, alkenyl, alkynyl, cycloalkyl, or aryl, or ═NRₕ where Rₕ is hydrogen, alkyl, alkenyl, alkynyl, cycloalkyl, or aryl, or is an amino acid residue.

4. The composition of claim 2 wherein the photosensitizer is post loaded onto the nanoparticle after nanoparticle formation.

5. The composition of claim 1 where the photosensitizer is selected from the group consisting of a chlorins, bacteriochlorins, pyropheophorbides, or mixtures thereof.

6. The composition of claim 1 wherein the imaging agent is a ¹²⁴I labeled compound.

7. The composition of claim 6 where the photosensitizer and imaging agent are the same compound.

8. The composition of claim 7 where the photosensitizer and imaging agent are the same compound and have the structural formula:

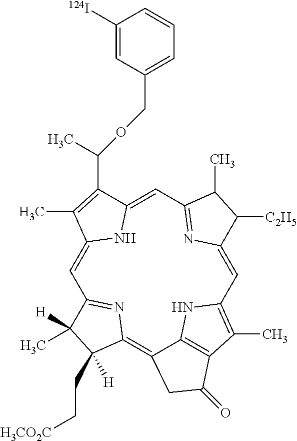 [SMILES: CCC1c2cc3[nH]c4c(c5nc(cc6[nH]c(cc(n2)C1C)c(C(C)OCc1cccc([124I])c1)c6C)[C@@H](C)[C@@H]5CCC(=O)OC)CC(=O)c4c3C]

9. The composition of claim 1 wherein the imaging agent is a PET imaging agent.

10. The composition of claim 9 wherein the nanoparticle contains a targeting moiety.

11. The composition of claim 10 wherein the targeting moiety is a peptide, folic acid or a carbohydrate.

12. A method for making PAA nanoparticle's containing a photosensitizer and an imaging agent by post loading a photosensitizer and a PET imaging agent onto a pre-prepared PAA nanoparticle.

13. The method of claim 10 where the photosensitizer has the structural formula: 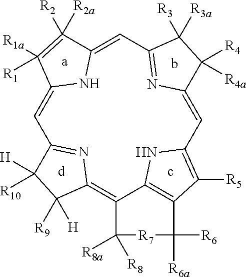
or a pharmaceutically acceptable derivative thereof, wherein:
R₁ and R₂ are each independently substituted or unsubstituted alkyl, substituted or unsubstituted alkenyl, —C(O)Rₐ or —COORₐ or —CH(CH₃)(ORₐ) or —CH(CH₃)(O(CH₂)ₙXRₐ) where Rₐ is hydrogen, substituted or unsubstituted alkyl, substituted or unsubstituted alkenyl, substituted or unsubstituted alkynyl, or substituted or unsubstituted cycloalkyl; where R₂ may be —CH═CH₂, —CH(OR₂₀)CH₃, —C(O)Me, —C(═NR₂₁)CH₃ or —CH(NHR₂₁)CH₃
where X is an aryl or heteroaryl group;
n is an integer of 0 to 6;
where R₂₀ is methyl, butyl, heptyl, docecyl or 3,5-bis(trifluoromethyl)-benzyl; and
R₂₁ is 3,5-bis(trifluoromethyl)benzyl;
R₁ₐ and R₂ₐ are each independently hydrogen or substituted or unsubstituted alkyl, or together form a covalent bond;
R₃ and R₄ are each independently hydrogen or substituted or unsubstituted alkyl;
R₃ₐ and R₄ₐ are each independently hydrogen or substituted or unsubstituted alkyl, or together form a covalent bond;
R₅ is hydrogen or substituted or unsubstituted alkyl;
R₆ and R₆ₐ are each independently hydrogen or substituted or unsubstituted alkyl, or together form ═O;
R₇ is a covalent bond, alkylene, azaalkyl, or azaaraalkyl or ═NR₂₀ where R₂₀ is 3,5-bis(tri-fluoromethyl)benzyl or —CH₂X—R¹ or —YR¹ where Y is an aryl or heteroaryl group;
R₈ and R₈ₐ are each independently hydrogen or substituted or unsubstituted alkyl or together form ═O;
R₉ and R₁₀ are each independently hydrogen, or substituted or unsubstituted alkyl and R₉ may be —CH₂CH₂COOR² where R² is an alkyl group that may optionally substituted with one or more fluorine atoms;
each of R₁-R₁₀, when substituted, is substituted with one or more substituents each independently selected from Q, where Q is alkyl, haloalkyl, halo, photosensitizereudohalo, or —COOR_{b} where R_{b} is hydrogen, alkyl, alkenyl, alkynyl, cycloalkyl, aryl, heteroaryl, araalkyl, or OR_{c} where R_{c} is hydrogen, alkyl, alkenyl, alkynyl, cycloalkyl, or aryl or CONR_{d}Rₑ where R_{d} and Rₑ are each independently hydrogen, alkyl, alkenyl, alkynyl, cycloalkyl, or aryl, or NR_{f}R_{g} where R_{f} and R_{g} are each independently hydrogen, alkyl, alkenyl, alkynyl, cycloalkyl, or aryl, or ═NRₕ where Rₕ is hydrogen, alkyl, alkenyl, alkynyl, cycloalkyl, or aryl, or is an amino acid residue and at least one of R₁-R₁₀ is substituted with ¹²⁴I;
each Q is independently unsubstituted or is substituted with one or more substituents each independently selected from Q₁, where Q₁ is alkyl, haloalkyl, halo, photosensitizereudohalo, or —COOR_{b} where R_{b} is hydrogen, alkyl, alkenyl, alkynyl, cycloalkyl, aryl, heteroaryl, araalkyl, or OR_{c} where R_{c} is hydrogen, alkyl, alkenyl, alkynyl, cycloalkyl, or aryl or CONR_{d}Rₑ where R_{d} and Rₑ are each independently hydrogen, alkyl, alkenyl, alkynyl, cycloalkyl, or aryl, or NR_{f}R_{g} where R_{f} and R_{g} are each independently hydrogen, alkyl, alkenyl, alkynyl, cycloalkyl, or aryl, or ═NRₕ where Rₕ is hydrogen, alkyl, alkenyl, alkynyl, cycloalkyl, or aryl, or is an amino acid residue.

14. The method of claim 13 where the photosensitizer is HPPH.

15. The composition of claim 1 where the photosensitizer is HPPH.

16. The composition of claim 1 where the imaging agent is HPPH substituted with a moiety containing ¹²⁴I.

17. The method of claim 13 where the imaging agent is HPPH substituted with a moiety containing ¹²⁴I.

18. The composition of claim 6 wherein the photosensitizer and imaging agent are postloaded onto pre-prepared PAA nanoparticles.

19. The composition of claim 18 where the numerical ratio of postloaded photosensitizer to imaging agent is from 1 to 1 to 10 to 1.

20. The composition of claim 19 where the numerical ratio of postloaded photosensitizer moieties to imaging agent is from 2 to 1 to 4 to 1.

21. The composition of claim 3 where the photosensitizer is a HPPH, purpurinimide having an absorbance between 680 and 720 nm, bacteriopurpurinimde having an absorbance between 780 and 800 nm or mixtures thereof.

22. A composition comprising a mixture of different PAA nanoparticles, at least one of which contains a postloaded photosensitizer and at least one of which contains a postloaded PET imaging agent.

23. A method for imaging and treatment of hyperproliferative tissue in an animal comprising:
a) injecting a composition according to claim 1 in an amount of 0.1 to 5.0 μmoles/kg,
b) imaging the animal by PET imaging to define and locate the hyperproliferative tissue, and
c) treating the defined and located hyperproliferative tissue with photodynamic therapy.

24. A method for imaging and treatment of hyperproliferative tissue in an animal comprising:
a) injecting a composition according to claim 2 in an amount of 0.5 to 3 μmoles/kg,
b) imaging the animal by PET imaging to define and locate the hyperproliferative tissue, and
c) treating the defined and located hyperproliferative tissue with photodynamic therapy.

25. A method for imaging and treatment of hyperproliferative tissue in an animal comprising:
a) injecting a composition according to claim 5 in an amount of 0.5 to 3.0 μmoles/kg,
b) imaging the animal by PET imaging to define and locate the hyperproliferative tissue, and
c) treating the defined and located hyperproliferative tissue with photodynamic therapy.

26. A method for imaging and treatment of hyperproliferative tissue in an animal comprising:
a) injecting a composition according to claim 8 in an amount of 0.5 to 3.0 μmoles/kg,
b) imaging the animal by PET imaging to define and locate the hyperproliferative tissue, and
c) treating the defined and located hyperproliferative tissue with photodynamic therapy.
